SEQUENCE LISTING (57 sequences)

SEQ ID NO: 1 - DNA, 64 bp, Homo sapiens
  source 1..64: mol_type = genomic DNA
ggaccctcgccagcgccacgcgcgccacgcccagggccaggagccagacaaaccccggcctgcg

SEQ ID NO: 2 - DNA, 64 bp, synthetic construct
  misc_feature 1..64: note = converted
  source 1..64: mol_type = other DNA
ggattttcgttagcgttacgcgcgttacgtttagggttaggagttagataaatttcggtttgcg

SEQ ID NO: 3 - DNA, 64 bp, synthetic construct
  misc_feature 1..64: note = converted
  source 1..64: mol_type = other DNA
cgcaaaccgaaatttatctaactcctaaccctaaacgtaacgcgcgtaacgctaacgaaaatcc

SEQ ID NO: 4 - DNA, 64 bp, synthetic construct
  misc_feature 1..64: note = converted
  source 1..64: mol_type = other DNA
aaaccctcgccaacgccacgcgcgccacgcccaaaaccaaaaaccaaacaaaccccgacctacg

SEQ ID NO: 5 - DNA, 64 bp, synthetic construct
  misc_feature 1..64: note = converted
  source 1..64: mol_type = other DNA
cgtaggtcggggtttgtttggtttttggttttgggcgtggcgcgcgtggcgttggcgagggttt

SEQ ID NO: 6 - DNA, 106 bp, Homo sapiens
  source 1..106: mol_type = genomic DNA
ggaccctcgccagcgccacgcgcgccacgcccagggccaggagccagacaaaccccggcctgcgcgcctgcagagcggcggctggagactggagccaccgcctcgc

SEQ ID NO: 7 - DNA, 106 bp, synthetic construct
  misc_feature 1..106: note = converted
  source 1..106: mol_type = other DNA
ggattttcgttagcgttacgcgcgttacgtttagggttaggagttagataaatttcggtttgcgcgtttgtagagcggcggttggagattggagttatcgtttcgt

SEQ ID NO: 8 - DNA, 106 bp, synthetic construct
  misc_feature 1..106: note = converted
  source 1..106: mol_type = other DNA
acgaaacgataactccaatctccaaccgccgctctacaaacgcgcaaaccgaaatttatctaactcctaaccctaaacgtaacgcgcgtaacgctaacgaaaatcc

SEQ ID NO: 9 - DNA, 106 bp, synthetic construct
  misc_feature 1..106: note = converted
  source 1..106: mol_type = other DNA
aaaccctcgccaacgccacgcgcgccacgcccaaaaccaaaaaccaaacaaaccccgacctacgcgcctacaaaacgacgactaaaaactaaaaccaccgcctcgc

SEQ ID NO: 10 - DNA, 106 bp, synthetic construct
  misc_feature 1..106: note = converted
  source 1..106: mol_type = other DNA
gcgaggcggtggttttagtttttagtcgtcgttttgtaggcgcgtaggtcggggtttgtttggtttttggttttgggcgtggcgcgcgtggcgttggcgagggttt

SEQ ID NO: 11 - DNA, 346 bp, Homo sapiens
  source 1..346: mol_type = genomic DNA
ggaccctcgccagcgccacgcgcgccacgcccagggccaggagccagacaaaccccggcctgcgcgcctgcagagcggcggctggagactggagccaccgcctcgcgggtgcagcagcacagcaggcacggttcggggagggattcggcatggccgcggggtaccttcccaggccagggccctggaggcaactggcacggcctggtccccctgctttgctgtattctgattccccaacccgctcccctgggctcagatctgtcctgtcctagggcggagccaggcggtcctgagggggagatgaatccttagaggagcgctgtccctatcccttgctccttctggc

SEQ ID NO: 12 - DNA, 346 bp, synthetic construct
  misc_feature 1..346: note = converted
  source 1..346: mol_type = other DNA
ggattttcgttagcgttacgcgcgttacgtttagggttaggagttagataaatttcggtttgcgcgtttgtagagcggcggttggagattggagttatcgtttcgcgggtgtagtagtatagtaggtacggttcggggagggattcggtatggtcgcggggtatttttttaggttagggttttggaggtaattggtacggtttggtttttttgttttgttgtattttgattttttaattcgtttttttgggtttagatttgttttgttttagggcggagttaggcggttttgagggggagatgaatttttagaggagcgttgtttttattttttgttttttttggt

SEQ ID NO: 13 - DNA, 346 bp, synthetic construct
  misc_feature 1..346: note = converted
  source 1..346: mol_type = other DNA
accaaaaaaaacaaaaaataaaaacaacgctcctctaaaaattcatctccccctcaaaaccgcctaactccgccctaaaacaaaacaaatctaaacccaaaaaaacgaattaaaaaatcaaaatacaacaaaacaaaaaaaccaaaccgtaccaattacctccaaaaccctaacctaaaaaaataccccgcgaccataccgaatccctccccgaaccgtacctactatactactacacccgcgaaacgataactccaatctccaaccgccgctctacaaacgcgcaaaccgaaatttatctaactcctaaccctaaacgtaacgcgcgtaacgctaacgaaaatcc

SEQ ID NO: 14 - DNA, 346 bp, synthetic construct
  misc_feature 1..346: note = converted
  source 1..346: mol_type = other DNA
aaaccctcgccaacgccacgcgcgccacgcccaaaaccaaaaaccaaacaaaccccgacctacgcgcctacaaaacgacgactaaaaactaaaaccaccgcctcgcgaatacaacaacacaacaaacacgattcgaaaaaaaattcgacataaccgcgaaataccttcccaaaccaaaaccctaaaaacaactaacacgacctaatccccctactttactatattctaattccccaacccgctcccctaaactcaaatctatcctatcctaaaacgaaaccaaacgatcctaaaaaaaaaataaatccttaaaaaaacgctatccctatcccttactccttctaac

SEQ ID NO: 15 - DNA, 346 bp, synthetic construct
  misc_feature 1..346: note = converted
  source 1..346: mol_type = other DNA
gttagaaggagtaagggatagggatagcgtttttttaaggatttattttttttttaggatcgtttggtttcgttttaggataggatagatttgagtttaggggagcgggttggggaattagaatatagtaaagtagggggattaggtcgtgttagttgtttttagggttttggtttgggaaggtatttcgcggttatgtcgaattttttttcgaatcgtgtttgttgtgttgttgtattcgcgaggcggtggttttagtttttagtcgtcgttttgtaggcgcgtaggtcggggtttgtttggtttttggttttgggcgtggcgcgcgtggcgttggcgagggttt

SEQ ID NO: 16 - DNA, 87 bp, Homo sapiens
  source 1..87: mol_type = genomic DNA
ggaccctcgccagcgccacgcgcgccacgcccagggccaggagccagacaaaccccggcctgcgcgcctgcagagcggcggctggag

SEQ ID NO: 17 - DNA, 87 bp, synthetic construct
  misc_feature 1..87: note = converted
  source 1..87: mol_type = other DNA
ggattttcgttagcgttacgcgcgttacgtttagggttaggagttagataaatttcggtttgcgcgtttgtagagcggcggttggag

SEQ ID NO: 18 - DNA, 87 bp, synthetic construct
  misc_feature 1..87: note = converted
  source 1..87: mol_type = other DNA
ctccaaccgccgctctacaaacgcgcaaaccgaaatttatctaactcctaaccctaaacgtaacgcgcgtaacgctaacgaaaatcc

SEQ ID NO: 19 - DNA, 87 bp, synthetic construct
  misc_feature 1..87: note = converted
  source 1..87: mol_type = other DNA
aaaccctcgccaacgccacgcgcgccacgcccaaaaccaaaaaccaaacaaaccccgacctacgcgcctacaaaacgacgactaaaa

SEQ ID NO: 20 - DNA, 87 bp, synthetic construct
  misc_feature 1..87: note = converted
  source 1..87: mol_type = other DNA
ttttagtcgtcgttttgtaggcgcgtaggtcggggtttgtttggtttttggttttgggcgtggcgcgcgtggcgttggcgagggttt

SEQ ID NO: 21 - DNA, 59 bp, Homo sapiens
  source 1..59: mol_type = genomic DNA
cgacccgctgcccaccagccatcatgtcggaccccgcggtcaacgcgcagctggatggg

SEQ ID NO: 22 - DNA, 59 bp, synthetic construct
  misc_feature 1..59: note = converted
  source 1..59: mol_type = other DNA
cgattcgttgtttattagttattatgtcggatttcgcggttaacgcgtagttggatggg

SEQ ID NO: 23 - DNA, 59 bp, synthetic construct
  misc_feature 1..59: note = converted
  source 1..59: mol_type = other DNA
cccatccaactacgcgttaaccgcgaaatccgacataataactaataaacaacgaatcg

SEQ ID NO: 24 - DNA, 59 bp, synthetic construct
  misc_feature 1..59: note = converted
  source 1..59: mol_type = other DNA
cgacccgctacccaccaaccatcatatcgaaccccgcgatcaacgcgcaactaaataaa

SEQ ID NO: 25 - DNA, 59 bp, synthetic construct
  misc_feature 1..59: note = converted
  source 1..59: mol_type = other DNA
tttatttagttgcgcgttgatcgcggggttcgatatgatggttggtgggtagcgggtcg

SEQ ID NO: 26 - DNA, 75 bp, Homo sapiens
  source 1..75: mol_type = genomic DNA
cgacccgctgcccaccagccatcatgtcggaccccgcggtcaacgcgcagctggatgggatcatttcggacttcg

SEQ ID NO: 27 - DNA, 75 bp, synthetic construct
  misc_feature 1..75: note = converted
  source 1..75: mol_type = other DNA
cgattcgttgtttattagttattatgtcggatttcgcggttaacgcgtagttggatgggattatttcggatttcg

SEQ ID NO: 28 - DNA, 75 bp, synthetic construct
  misc_feature 1..75: note = converted
  source 1..75: mol_type = other DNA
cgaaatccgaaataatcccatccaactacgcgttaaccgcgaaatccgacataataactaataaacaacgaatcg

SEQ ID NO: 29 - DNA, 75 bp, synthetic construct
  misc_feature 1..75: note = converted
  source 1..75: mol_type = other DNA
cgacccgctacccaccaaccatcatatcgaaccccgcgatcaacgcgcaactaaataaaatcatttcgaacttcg

SEQ ID NO: 30 - DNA, 75 bp, synthetic construct
  misc_feature 1..75: note = converted
  source 1..75: mol_type = other DNA
cgaagttcgaaatgattttatttagttgcgcgttgatcgcggggttcgatatgatggttggtgggtagcgggtcg

SEQ ID NO: 31 - DNA, 1819 bp, Homo sapiens
  source 1..1819: mol_type = genomic DNA
gcgtcgcccgtccctggcttctctgacagccgtgttccatccccgccctgtgccccttctcccggacagtgccttctccagggctcacccaggagggtgcagcggtggcccccggggcggtggtcgtggtgggggtgttagctgcaggggtgccctcggtgggtgggagttggtggcctctcgctggtgccatgggactcgcatgttcgccctgcgcccctcggctcttgagcccacaggccgggatcctgcctgccagccgcgtgcgctgccgtttaacccttgcaggcgcagagcgcgcggcggcggtgacagagaactttgtttggctgcccaaatacagcctcctgcagaaggaccctgcgcccggggaaggggaggaatctcttcccctctgggcgcccgccctcctcgccatggcccggcctccacatccgcccacatctggccgcagcggggcgcccggggggaggggctgaggccgcgtctctcgccgtcccctgggcgcgggccaggcggggaggaggggggcgctccggtcgtgtgcccaggactgtcccccagcggccactcgggccccagccccccaggcctggccttgacaggcgggcggagcagccagtgcgagacagggaggccggtgcgggtgcgggaacctgatccgcccgggaggcgggggcggggcgggggcgcagcgcgcggggaggggccggcgcccgccttcctcccccattcattcagctgagccagggggcctaggggctcctccggcggctagctctgcactgcaggagcgcgggcgcggcgccccagccagcgcgcagggcccgggccccgccgggggcgcttcctcgccgctgccctccgcgcgacccgctgcccaccagccatcatgtcggaccccgcggtcaacgcgcagctggatgggatcatttcggacttcgaaggtgggtgctgggctggctgctgcggccgcggacgtgctggagaggaccctgcgggtgggcctggcgcgggacgggggtgcgctgaggggagacgggagtgcgctgaggggagacgggacccctaatccaggcgccctcccgctgagagcgccgcgcgcccccggccccgtgcccgcgccgcctacgtgggggaccctgttaggggcacccgcgtagaccctgcgcgccctcacaggaccctgtgctcgttctgcgcactgccgcctgggtttccttccttttattgttgtttgtgtttgccaagcgacagcgacctcctcgagggctcgcgaggctgcctcggaactctccaggacgcacagtttcactctgggaaatccatcggtcccctccctttggctctccccggcggctctcgggccccgcttggacccggcaacgggatagggaggtcgttcctcacctccgactgagtggacagccgcgtcctgctcgggtggacagccctcccctcccccacgccagtttcggggccgccaagttgtgcagcccgtgggccgggagcaccgaacggacacagcccaggtcgtggcagggtctagagtgggatgtcccatggcccccatccaggcctggggatatcctcatccgcctcccagaatcgggccgtgggggacagaaggggcctgcgtgcgggcagggagagtattttggctctctcctgtcttcggggtttacaaagtgtgttgggacttgcggggctgctctgtccaagcctgggtctggcgtccgcgtctctgagcctgtgagtgcgtgcgctttcctgcgtcctcttgactgccggtgctggggctctgcgtcctgcgtccgcg

SEQ ID NO: 32 - DNA, 1819 bp, synthetic construct
  misc_feature 1..1819: note = converted
  source 1..1819: mol_type = other DNA
gcgtcgttcgtttttggtttttttgatagtcgtgttttattttcgttttgtgttttttttttcggatagtgttttttttagggtttatttaggagggtgtagcggtggttttcggggcggtggtcgtggtgggggtgttagttgtaggggtgttttcggtgggtgggagttggtggtttttcgttggtgttatgggattcgtatgttcgttttgcgtttttcggtttttgagtttataggtcgggattttgtttgttagtcgcgtgcgttgtcgtttaatttttgtaggcgtagagcgcgcggcggcggtgatagagaattttgtttggttgtttaaatatagttttttgtagaaggattttgcgttcggggaaggggaggaatttttttttttttgggcgttcgtttttttcgttatggttcggtttttatattcgtttatatttggtcgtagcggggcgttcggggggaggggttgaggtcgcgtttttcgtcgttttttgggcgcgggttaggcggggaggaggggggcgtttcggtcgtgtgtttaggattgttttttagcggttattcgggttttagttttttaggtttggttttgataggcgggcggagtagttagtgcgagatagggaggtcggtgcgggtgcgggaatttgattcgttcgggaggcgggggcggggcgggggcgtagcgcgcggggaggggtcggcgttcgttttttttttttatttatttagttgagttagggggtttaggggttttttcggcggttagttttgtattgtaggagcgcgggcgcggcgttttagttagcgcgtagggttcgggtttcgtcgggggcgttttttcgtcgttgtttttcgcgcgattcgttgtttattagttattatgtcggatttcgcggttaacgcgtagttggatgggattatttcggatttcgaaggtgggtgttgggttggttgttgcggtcgcggacgtgttggagaggattttgcgggtgggtttggcgcgggacgggggtgcgttgaggggagacgggagtgcgttgaggggagacgggatttttaatttaggcgttttttcgttgagagcgtcgcgcgttttcggtttcgtgttcgcgtcgtttacgtgggggattttgttaggggtattcgcgtagattttgcgcgtttttataggattttgtgttcgttttgcgtattgtcgtttgggtttttttttttttattgttgtttgtgtttgttaagcgatagcgattttttcgagggttcgcgaggttgtttcggaattttttaggacgtatagttttattttgggaaatttatcggttttttttttttggtttttttcggcggttttcgggtttcgtttggattcggtaacgggatagggaggtcgttttttattttcgattgagtggatagtcgcgttttgttcgggtggatagttttttttttttttacgttagtttcggggtcgttaagttgtgtagttcgtgggtcgggagtatcgaacggatatagtttaggtcgtggtagggtttagagtgggatgttttatggtttttatttaggtttggggatatttttattcgttttttagaatcgggtcgtgggggatagaaggggtttgcgtgcgggtagggagagtattttggtttttttttgttttcggggtttataaagtgtgttgggatttgcggggttgttttgtttaagtttgggtttggcgttcgcgtttttgagtttgtgagtgcgtgcgtttttttgcgtttttttgattgtcggtgttggggttttgcgttttgcgttcgcg

SEQ ID NO: 33 - DNA, 1819 bp, synthetic construct
  misc_feature 1..1819: note = converted
  source 1..1819: mol_type = other DNA
cgcgaacgcaaaacgcaaaaccccaacaccgacaatcaaaaaaacgcaaaaaaacgcacgcactcacaaactcaaaaacgcgaacgccaaacccaaacttaaacaaaacaaccccgcaaatcccaacacactttataaaccccgaaaacaaaaaaaaaccaaaatactctccctacccgcacgcaaaccccttctatcccccacgacccgattctaaaaaacgaataaaaatatccccaaacctaaataaaaaccataaaacatcccactctaaaccctaccacgacctaaactatatccgttcgatactcccgacccacgaactacacaacttaacgaccccgaaactaacgtaaaaaaaaaaaaaactatccacccgaacaaaacgcgactatccactcaatcgaaaataaaaaacgacctccctatcccgttaccgaatccaaacgaaacccgaaaaccgccgaaaaaaaccaaaaaaaaaaaaccgataaatttcccaaaataaaactatacgtcctaaaaaattccgaaacaacctcgcgaaccctcgaaaaaatcgctatcgcttaacaaacacaaacaacaataaaaaaaaaaaaacccaaacgacaatacgcaaaacgaacacaaaatcctataaaaacgcgcaaaatctacgcgaatacccctaacaaaatcccccacgtaaacgacgcgaacacgaaaccgaaaacgcgcgacgctctcaacgaaaaaacgcctaaattaaaaatcccgtctcccctcaacgcactcccgtctcccctcaacgcacccccgtcccgcgccaaacccacccgcaaaatcctctccaacacgtccgcgaccgcaacaaccaacccaacacccaccttcgaaatccgaaataatcccatccaactacgcgttaaccgcgaaatccgacataataactaataaacaacgaatcgcgcgaaaaacaacgacgaaaaaacgcccccgacgaaacccgaaccctacgcgctaactaaaacgccgcgcccgcgctcctacaatacaaaactaaccgccgaaaaaacccctaaaccccctaactcaactaaataaataaaaaaaaaaaacgaacgccgacccctccccgcgcgctacgcccccgccccgcccccgcctcccgaacgaatcaaattcccgcacccgcaccgacctccctatctcgcactaactactccgcccgcctatcaaaaccaaacctaaaaaactaaaacccgaataaccgctaaaaaacaatcctaaacacacgaccgaaacgcccccctcctccccgcctaacccgcgcccaaaaaacgacgaaaaacgcgacctcaacccctccccccgaacgccccgctacgaccaaatataaacgaatataaaaaccgaaccataacgaaaaaaacgaacgcccaaaaaaaaaaaaattcctccccttccccgaacgcaaaatccttctacaaaaaactatatttaaacaaccaaacaaaattctctatcaccgccgccgcgcgctctacgcctacaaaaattaaacgacaacgcacgcgactaacaaacaaaatcccgacctataaactcaaaaaccgaaaaacgcaaaacgaacatacgaatcccataacaccaacgaaaaaccaccaactcccacccaccgaaaacacccctacaactaacacccccaccacgaccaccgccccgaaaaccaccgctacaccctcctaaataaaccctaaaaaaaacactatccgaaaaaaaaaacacaaaacgaaaataaaacacgactatcaaaaaaaccaaaaacgaacgacgc

SEQ ID NO: 34 - DNA, 1819 bp, synthetic construct
  misc_feature 1..1819: note = converted
  source 1..1819: mol_type = other DNA
acgtcgcccgtccctaacttctctaacaaccgtattccatccccgccctataccccttctcccgaacaataccttctccaaaactcacccaaaaaaatacaacgataacccccgaaacgataatcgtaataaaaatattaactacaaaaataccctcgataaataaaaattaataacctctcgctaataccataaaactcgcatattcgccctacgcccctcgactcttaaacccacaaaccgaaatcctacctaccaaccgcgtacgctaccgtttaacccttacaaacgcaaaacgcgcgacgacgataacaaaaaactttatttaactacccaaatacaacctcctacaaaaaaaccctacgcccgaaaaaaaaaaaaaatctcttcccctctaaacgcccgccctcctcgccataacccgacctccacatccgcccacatctaaccgcaacgaaacgcccgaaaaaaaaaactaaaaccgcgtctctcgccgtcccctaaacgcgaaccaaacgaaaaaaaaaaaaacgctccgatcgtatacccaaaactatcccccaacgaccactcgaaccccaaccccccaaacctaaccttaacaaacgaacgaaacaaccaatacgaaacaaaaaaaccgatacgaatacgaaaacctaatccgcccgaaaaacgaaaacgaaacgaaaacgcaacgcgcgaaaaaaaaccgacgcccgccttcctcccccattcattcaactaaaccaaaaaacctaaaaactcctccgacgactaactctacactacaaaaacgcgaacgcgacgccccaaccaacgcgcaaaacccgaaccccgccgaaaacgcttcctcgccgctaccctccgcgcgacccgctacccaccaaccatcatatcgaaccccgcgatcaacgcgcaactaaataaaatcatttcgaacttcgaaaataaatactaaactaactactacgaccgcgaacgtactaaaaaaaaccctacgaataaacctaacgcgaaacgaaaatacgctaaaaaaaaacgaaaatacgctaaaaaaaaacgaaacccctaatccaaacgccctcccgctaaaaacgccgcgcgcccccgaccccgtacccgcgccgcctacgtaaaaaaccctattaaaaacacccgcgtaaaccctacgcgccctcacaaaaccctatactcgttctacgcactaccgcctaaatttccttccttttattattatttatatttaccaaacgacaacgacctcctcgaaaactcgcgaaactacctcgaaactctccaaaacgcacaatttcactctaaaaaatccatcgatcccctccctttaactctccccgacgactctcgaaccccgcttaaacccgacaacgaaataaaaaaatcgttcctcacctccgactaaataaacaaccgcgtcctactcgaataaacaaccctcccctcccccacgccaatttcgaaaccgccaaattatacaacccgtaaaccgaaaacaccgaacgaacacaacccaaatcgtaacaaaatctaaaataaaatatcccataacccccatccaaacctaaaaatatcctcatccgcctcccaaaatcgaaccgtaaaaaacaaaaaaaacctacgtacgaacaaaaaaaatattttaactctctcctatcttcgaaatttacaaaatatattaaaacttacgaaactactctatccaaacctaaatctaacgtccgcgtctctaaacctataaatacgtacgctttcctacgtcctcttaactaccgatactaaaactctacgtcctacgtccgcg

SEQ ID NO: 35 - DNA, 1819 bp, synthetic construct
  misc_feature 1..1819: note = converted
  source 1..1819: mol_type = other DNA
cgcggacgtaggacgtagagttttagtatcggtagttaagaggacgtaggaaagcgtacgtatttataggtttagagacgcggacgttagatttaggtttggatagagtagtttcgtaagttttaatatattttgtaaatttcgaagataggagagagttaaaatattttttttgttcgtacgtaggttttttttgttttttacggttcgattttgggaggcggatgaggatatttttaggtttggatgggggttatgggatattttattttagattttgttacgatttgggttgtgttcgttcggtgttttcggtttacgggttgtataatttggcggtttcgaaattggcgtgggggaggggagggttgtttattcgagtaggacgcggttgtttatttagtcggaggtgaggaacgatttttttatttcgttgtcgggtttaagcggggttcgagagtcgtcggggagagttaaagggaggggatcgatggattttttagagtgaaattgtgcgttttggagagtttcgaggtagtttcgcgagttttcgaggaggtcgttgtcgtttggtaaatataaataataataaaaggaaggaaatttaggcggtagtgcgtagaacgagtatagggttttgtgagggcgcgtagggtttacgcgggtgtttttaatagggttttttacgtaggcggcgcgggtacggggtcgggggcgcgcggcgtttttagcgggagggcgtttggattaggggtttcgtttttttttagcgtattttcgtttttttttagcgtattttcgtttcgcgttaggtttattcgtagggttttttttagtacgttcgcggtcgtagtagttagtttagtatttattttcgaagttcgaaatgattttatttagttgcgcgttgatcgcggggttcgatatgatggttggtgggtagcgggtcgcgcggagggtagcggcgaggaagcgttttcggcggggttcgggttttgcgcgttggttggggcgtcgcgttcgcgtttttgtagtgtagagttagtcgtcggaggagtttttaggttttttggtttagttgaatgaatgggggaggaaggcgggcgtcggttttttttcgcgcgttgcgttttcgtttcgttttcgtttttcgggcggattaggttttcgtattcgtatcggtttttttgtttcgtattggttgtttcgttcgtttgttaaggttaggtttggggggttggggttcgagtggtcgttgggggatagttttgggtatacgatcggagcgtttttttttttttcgtttggttcgcgtttaggggacggcgagagacgcggttttagttttttttttcgggcgtttcgttgcggttagatgtgggcggatgtggaggtcgggttatggcgaggagggcgggcgtttagaggggaagagattttttttttttttcgggcgtagggtttttttgtaggaggttgtatttgggtagttaaataaagttttttgttatcgtcgtcgcgcgttttgcgtttgtaagggttaaacggtagcgtacgcggttggtaggtaggatttcggtttgtgggtttaagagtcgaggggcgtagggcgaatatgcgagttttatggtattagcgagaggttattaatttttatttatcgagggtatttttgtagttaatatttttattacgattatcgtttcgggggttatcgttgtatttttttgggtgagttttggagaaggtattgttcgggagaaggggtatagggcggggatggaatacggttgttagagaagttagggacgggcgacgt

SEQ ID NO: 36 - DNA, 1062 bp, Homo sapiens
  source 1..1062: mol_type = genomic DNA
aggggaggaatctcttcccctctgggcgcccgccctcctcgccatggcccggcctccacatccgcccacatctggccgcagcggggcgcccggggggaggggctgaggccgcgtctctcgccgtcccctgggcgcgggccaggcggggaggaggggggcgctccggtcgtgtgcccaggactgtcccccagcggccactcgggccccagccccccaggcctggccttgacaggcgggcggagcagccagtgcgagacagggaggccggtgcgggtgcgggaacctgatccgcccgggaggcgggggcggggcgggggcgcagcgcgcggggaggggccggcgcccgccttcctcccccattcattcagctgagccagggggcctaggggctcctccggcggctagctctgcactgcaggagcgcgggcgcggcgccccagccagcgcgcagggcccgggccccgccgggggcgcttcctcgccgctgccctccgcgcgacccgctgcccaccagccatcatgtcggaccccgcggtcaacgcgcagctggatgggatcatttcggacttcgaaggtgggtgctgggctggctgctgcggccgcggacgtgctggagaggaccctgcgggtgggcctggcgcgggacgggggtgcgctgaggggagacgggagtgcgctgaggggagacgggacccctaatccaggcgccctcccgctgagagcgccgcgcgcccccggccccgtgcccgcgccgcctacgtgggggaccctgttaggggcacccgcgtagaccctgcgcgccctcacaggaccctgtgctcgttctgcgcactgccgcctgggtttccttccttttattgttgtttgtgtttgccaagcgacagcgacctcctcgagggctcgcgaggctgcctcggaactctccaggacgcacagtttcactctgggaaatccatcggtcccctccctttggctctccccggcggctctcgggccccgcttggacccggcaacgggatagggaggtcgttcctcacctccgactgagtggacagccgcgtc

SEQ ID NO: 37 - DNA, 1062 bp, synthetic construct
  misc_feature 1..1062: note = converted
  source 1..1062: mol_type = other DNA
aggggaggaatttttttttttttgggcgttcgtttttttcgttatggttcggtttttatattcgtttatatttggtcgtagcggggcgttcggggggaggggttgaggtcgcgtttttcgtcgttttttgggcgcgggttaggcggggaggaggggggcgtttcggtcgtgtgtttaggattgttttttagcggttattcgggttttagttttttaggtttggttttgataggcgggcggagtagttagtgcgagatagggaggtcggtgcgggtgcgggaatttgattcgttcgggaggcgggggcggggcgggggcgtagcgcgcggggaggggtcggcgttcgttttttttttttatttatttagttgagttagggggtttaggggttttttcggcggttagttttgtattgtaggagcgcgggcgcggcgttttagttagcgcgtagggttcgggtttcgtcgggggcgttttttcgtcgttgtttttcgcgcgattcgttgtttattagttattatgtcggatttcgcggttaacgcgtagttggatgggattatttcggatttcgaaggtgggtgttgggttggttgttgcggtcgcggacgtgttggagaggattttgcgggtgggtttggcgcgggacgggggtgcgttgaggggagacgggagtgcgttgaggggagacgggatttttaatttaggcgttttttcgttgagagcgtcgcgcgttttcggtttcgtgttcgcgtcgtttacgtgggggattttgttaggggtattcgcgtagattttgcgcgtttttataggattttgtgttcgttttgcgtattgtcgtttgggtttttttttttttattgttgtttgtgtttgttaagcgatagcgattttttcgagggttcgcgaggttgtttcggaattttttaggacgtatagttttattttgggaaatttatcggttttttttttttggtttttttcggcggttttcgggtttcgtttggattcggtaacgggatagggaggtcgttttttattttcgattgagtggatagtcgcgtt

SEQ ID NO: 38 - DNA, 1062 bp, synthetic construct
  misc_feature 1..1062: note = converted
  source 1..1062: mol_type = other DNA
aacgcgactatccactcaatcgaaaataaaaaacgacctccctatcccgttaccgaatccaaacgaaacccgaaaaccgccgaaaaaaaccaaaaaaaaaaaaccgataaatttcccaaaataaaactatacgtcctaaaaaattccgaaacaacctcgcgaaccctcgaaaaaatcgctatcgcttaacaaacacaaacaacaataaaaaaaaaaaaacccaaacgacaatacgcaaaacgaacacaaaatcctataaaaacgcgcaaaatctacgcgaatacccctaacaaaatcccccacgtaaacgacgcgaacacgaaaccgaaaacgcgcgacgctctcaacgaaaaaacgcctaaattaaaaatcccgtctcccctcaacgcactcccgtctcccctcaacgcacccccgtcccgcgccaaacccacccgcaaaatcctctccaacacgtccgcgaccgcaacaaccaacccaacacccaccttcgaaatccgaaataatcccatccaactacgcgttaaccgcgaaatccgacataataactaataaacaacgaatcgcgcgaaaaacaacgacgaaaaaacgcccccgacgaaacccgaaccctacgcgctaactaaaacgccgcgcccgcgctcctacaatacaaaactaaccgccgaaaaaacccctaaaccccctaactcaactaaataaataaaaaaaaaaaacgaacgccgacccctccccgcgcgctacgcccccgccccgcccccgcctcccgaacgaatcaaattcccgcacccgcaccgacctccctatctcgcactaactactccgcccgcctatcaaaaccaaacctaaaaaactaaaacccgaataaccgctaaaaaacaatcctaaacacacgaccgaaacgcccccctcctccccgcctaacccgcgcccaaaaaacgacgaaaaacgcgacctcaacccctccccccgaacgccccgctacgaccaaatataaacgaatataaaaaccgaaccataacgaaaaaaacgaacgcccaaaaaaaaaaaaattcctcccct

SEQ ID NO: 39 - DNA, 1062 bp, synthetic construct
  misc_feature 1..1062: note = converted
  source 1..1062: mol_type = other DNA
aaaaaaaaaatctcttcccctctaaacgcccgccctcctcgccataacccgacctccacatccgcccacatctaaccgcaacgaaacgcccgaaaaaaaaaactaaaaccgcgtctctcgccgtcccctaaacgcgaaccaaacgaaaaaaaaaaaaacgctccgatcgtatacccaaaactatcccccaacgaccactcgaaccccaaccccccaaacctaaccttaacaaacgaacgaaacaaccaatacgaaacaaaaaaaccgatacgaatacgaaaacctaatccgcccgaaaaacgaaaacgaaacgaaaacgcaacgcgcgaaaaaaaaccgacgcccgccttcctcccccattcattcaactaaaccaaaaaacctaaaaactcctccgacgactaactctacactacaaaaacgcgaacgcgacgccccaaccaacgcgcaaaacccgaaccccgccgaaaacgcttcctcgccgctaccctccgcgcgacccgctacccaccaaccatcatatcgaaccccgcgatcaacgcgcaactaaataaaatcatttcgaacttcgaaaataaatactaaactaactactacgaccgcgaacgtactaaaaaaaaccctacgaataaacctaacgcgaaacgaaaatacgctaaaaaaaaacgaaaatacgctaaaaaaaaacgaaacccctaatccaaacgccctcccgctaaaaacgccgcgcgcccccgaccccgtacccgcgccgcctacgtaaaaaaccctattaaaaacacccgcgtaaaccctacgcgccctcacaaaaccctatactcgttctacgcactaccgcctaaatttccttccttttattattatttatatttaccaaacgacaacgacctcctcgaaaactcgcgaaactacctcgaaactctccaaaacgcacaatttcactctaaaaaatccatcgatcccctccctttaactctccccgacgactctcgaaccccgcttaaacccgacaacgaaataaaaaaatcgttcctcacctccgactaaataaacaaccgcgtc

SEQ ID NO: 40 - DNA, 1062 bp, synthetic construct
  misc_feature 1..1062: note = converted
  source 1..1062: mol_type = other DNA
gacgcggttgtttatttagtcggaggtgaggaacgatttttttatttcgttgtcgggtttaagcggggttcgagagtcgtcggggagagttaaagggaggggatcgatggattttttagagtgaaattgtgcgttttggagagtttcgaggtagtttcgcgagttttcgaggaggtcgttgtcgtttggtaaatataaataataataaaaggaaggaaatttaggcggtagtgcgtagaacgagtatagggttttgtgagggcgcgtagggtttacgcgggtgtttttaatagggttttttacgtaggcggcgcgggtacggggtcgggggcgcgcggcgtttttagcgggagggcgtttggattaggggtttcgtttttttttagcgtattttcgtttttttttagcgtattttcgtttcgcgttaggtttattcgtagggttttttttagtacgttcgcggtcgtagtagttagtttagtatttattttcgaagttcgaaatgattttatttagttgcgcgttgatcgcggggttcgatatgatggttggtgggtagcgggtcgcgcggagggtagcggcgaggaagcgttttcggcggggttcgggttttgcgcgttggttggggcgtcgcgttcgcgtttttgtagtgtagagttagtcgtcggaggagtttttaggttttttggtttagttgaatgaatgggggaggaaggcgggcgtcggttttttttcgcgcgttgcgttttcgtttcgttttcgtttttcgggcggattaggttttcgtattcgtatcggtttttttgtttcgtattggttgtttcgttcgtttgttaaggttaggtttggggggttggggttcgagtggtcgttgggggatagttttgggtatacgatcggagcgtttttttttttttcgtttggttcgcgtttaggggacggcgagagacgcggttttagttttttttttcgggcgtttcgttgcggttagatgtgggcggatgtggaggtcgggttatggcgaggagggcgggcgtttagaggggaagagatttttttttt

SEQ ID NO: 41 - DNA, 68 bp, Homo sapiens
  source 1..68: mol_type = genomic DNA
ctgcccaccagccatcatgtcggaccccgcggtcaacgcgcagctggatgggatcatttcggacttcg

SEQ ID NO: 42 - DNA, 68 bp, synthetic construct
  misc_feature 1..68: note = converted
  source 1..68: mol_type = other DNA
ttgtttattagttattatgtcggatttcgcggttaacgcgtagttggatgggattatttcggatttcg

SEQ ID NO: 43 - DNA, 68 bp, synthetic construct
  misc_feature 1..68: note = converted
  source 1..68: mol_type = other DNA
cgaaatccgaaataatcccatccaactacgcgttaaccgcgaaatccgacataataactaataaacaa

SEQ ID NO: 44 - DNA, 68 bp, synthetic construct
  misc_feature 1..68: note = converted
  source 1..68: mol_type = other DNA
ctacccaccaaccatcatatcgaaccccgcgatcaacgcgcaactaaataaaatcatttcgaacttcg

SEQ ID NO: 45 - DNA, 68 bp, synthetic construct
  misc_feature 1..68: note = converted
  source 1..68: mol_type = other DNA
cgaagttcgaaatgattttatttagttgcgcgttgatcgcggggttcgatatgatggttggtgggtag

SEQ ID NO: 46 - DNA, 18 bp, synthetic construct
  misc_feature 1..18: note = primer
  source 1..18: mol_type = other DNA
ggattttcgttagcgtta

SEQ ID NO: 47 - DNA, 18 bp, synthetic construct
  misc_feature 1..18: note = primer
  source 1..18: mol_type = other DNA
cgcaaaccgaaatttatc

SEQ ID NO: 48 - DNA, 15 bp, synthetic construct
  misc_feature 1..15: note = primer
  source 1..15: mol_type = other DNA
aaaccctcgccaacg

SEQ ID NO: 49 - DNA, 22 bp, synthetic construct
  misc_feature 1..22: note = primer
  source 1..22: mol_type = other DNA
ttttagtcgtcgttttgtaggc

SEQ ID NO: 50 - DNA, 24 bp, synthetic construct
  misc_feature 1..24: note = primer
  source 1..24: mol_type = other DNA
cgattcgttgtttattagttatta

SEQ ID NO: 51 - DNA, 16 bp, synthetic construct
  misc_feature 1..16: note = primer
  source 1..16: mol_type = other DNA
cccatccaactacgcg

SEQ ID NO: 52 - DNA, 22 bp, synthetic construct
  misc_feature 1..22: note = primer
  source 1..22: mol_type = other DNA
cgaagttcgaaatgattttatt

SEQ ID NO: 53 - DNA, 21 bp, synthetic construct
  misc_feature 1..21: note = primer
  source 1..21: mol_type = other DNA
ctacccaccaaccatcatatc

SEQ ID NO: 54 - DNA, 23 bp, synthetic construct
  misc_feature 1..23: note = primer
  source 1..23: mol_type = other DNA
cgcgttacgtttagggttaggag

SEQ ID NO: 55 - DNA, 20 bp, synthetic construct
  misc_feature 1..20: note = primer
  source 1..20: mol_type = other DNA
cacgcgcgccacgcccaaaa

SEQ ID NO: 56 - DNA, 18 bp, synthetic construct
  misc_feature 1..18: note = primer
  source 1..18: mol_type = other DNA
taaccgcgaaatccgaca

SEQ ID NO: 57 - DNA, 17 bp, synthetic construct
  misc_feature 1..17: note = primer
  source 1..17: mol_type = other DNA
cgttgatcgcggggttc

(19) United States
(12) Patent Application Publication
(10) Pub. No.: US 2025/0129427 A1
(43) Pub. Date: Apr. 24, 2025

(54) IMPROVED METHODS FOR DETECTING COLORECTAL CANCER

(71) Applicant: Epigenomics AG, Berlin (DE)

(72) Inventors: Denise KOTTWITZ, Berlin (DE); Oliver HASINGER, Berlin (DE); Preeti RATHI, Berlin (DE); Thomas KÖNIG, Berlin (DE); Gunter WEISS, Berlin (DE); Anne SCHLEGEL, Berlin (DE); Jörn LEWIN, Berlin (DE)

(73) Assignee: New Day Diagnostics LLC, Knoxville, TN (US)

(21) Appl. No.: 18/682,779

(22) PCT Filed: Aug. 8, 2023
(86) PCT No.: PCT/EP2022/072276

§ 371 Date: Feb. 9, 2024

(30) Foreign Application Priority Data
Aug. 9, 2021 (EP) 21190355.4

(51) Int. Cl. C12Q 1/6886

(52) U.S. Cl. CPC C12Q 1/6886; C12Q 2600/154

(57) ABSTRACT

The present invention relates to the field of pharmacogenomics and in particular to detecting the presence or absence of methylated ANKRD13B and/or SEPTIN9 DNA derived from a tumor in blood or blood-derived samples or in other body fluids that contain DNA released from a tumor. This detection is useful for a minimally or non-invasive as well as early and reliable diagnosis of colorectal cancer. The invention provides methods and oligonucleotides suitable for this purpose.

### FIELD OF THE INVENTION

The present invention relates to the field of pharmacogenomics and in particular to detecting the presence or absence of methylated ANKRD13B and/or SEPTIN9 DNA derived from a tumor in blood or blood-derived samples or in other body fluids that contain DNA released from a tumor. This detection is useful for a minimally or non-invasive as well as early and reliable diagnosis of colorectal cancer. The invention provides methods and oligonucleotides suitable for this purpose.

### BACKGROUND OF THE INVENTION

Colorectal cancer (CRC) encompasses tumors originating from the colon and rectum. It is the third most common cancer worldwide, but the second most common cancer killer. When colorectal cancer is found at an early stage, the 5-year relative survival rate is about 90%. At advanced stages, however, colorectal cancer survival rates are very low. Conventional CRC screening involved either visual exams or stool-based tests. Visual exams look at the structure of the colon and rectum for abnormal areas using a scope put into the rectum (e.g. colonoscopy or sigmoidoscopy) or non-invasive imaging techniques (e.g. x-ray or CR colonography (virtual colonoscopy)). Stool tests such as FIT (Fecal immunochemical test) or gFOBT (Guaiac-based fecal occult blood test) usually detect blood or polyps in stool samples. Stool tests have relatively low sensitivity and specificity and are also problematic with regard to participants' compliance, satisfaction and intention to be re-screened. Invasive visual exams are uncomfortable and incur a risk of bleeding, tears and infection. Therefore, they are often avoided by at-risk subjects. Non-invasive imaging techniques expose the subjects to radiation and often miss small polyps.

DNA methylation patterns are largely modified in cancer cells and can therefore be used to distinguish cancer cells from normal tissues. As such, DNA methylation patterns are being used to diagnose all sorts of cancers. One of the challenges is diagnosing a cancer as early as possible, because a less advanced cancer, which has smaller tumor size and less cancer cells, releases less free circulating tumor DNA that is available for methylation detection. Thus, there is an ongoing need to provide methylation detection methods with increased sensitivity and specificity.

Methylated ANKRD13B and SEPTIN9 DNA has been used previously for CRC detection (WO 2017/129716 and WO 2006/113466, respectively). The present invention provides improved methods for detecting CRC using mANKRD13B (methylated ANKRD13B DNA) and mSEPTIN9 (methylated SEPTIN9 DNA) as markers.

### SUMMARY OF THE INVENTION

In a first aspect, the present invention relates to a method for detecting DNA methylation (i) at one or more CpG dinucleotides within genomic DNA having a sequence comprised in SEQ ID NO: 11 (mANKRD13B target region) and optionally (ii) at one or more CpG dinucleotides within genomic DNA having a sequence comprised in SEQ ID NO: 31 (mSEPTIN9 target region) in a biological sample of a subject.

In a second aspect, the present invention relates to a method for detecting colorectal cancer (CRC) in a subject, comprising detecting DNA methylation according to the first aspect, wherein
(1), if the mANKRD13B target region and not the mSEPTIN9 target region is examined, the presence of detected methylated genomic DNA indicates the presence of CRC and the absence of detected methylated genomic DNA indicates the absence of CRC, and
(2), if both the mANKRD13B target region and the mSEPTIN9 target region are examined, the presence of detected methylated genomic DNA of at least one of the target regions indicates the presence of CRC.

In a third aspect, the present invention relates to a method for monitoring a subject suspected of having CRC, having an increased risk of developing CRC, or who has had CRC, comprising detecting DNA methylation according to the first aspect repeatedly, wherein
(1), if the mANKRD13B target region and not the mSEPTIN9 target region is examined, the presence of detected methylated genomic DNA indicates the presence of CRC and the absence of detected methylated genomic DNA indicates the absence of CRC, and
(2), if both the mANKRD13B target region and the mSEPTIN9 target region are examined, the presence of detected methylated genomic DNA of at least one of the target regions indicates the presence of CRC.

In a fourth aspect, the present invention relates to a kit comprising (i) a primer pair consisting of a forward primer oligonucleotide comprising a sequence that is substantially identical to a stretch of contiguous nucleotides of SEQ ID NO: 12 or 15, and a reverse primer oligonucleotide comprising a sequence that is substantially identical to a stretch of contiguous nucleotides of SEQ ID NO: 13 or 14, respectively, wherein the primer pair is suitable for amplification of a sequence comprising one or more CpG dinucleotides of SEQ ID NO: 12 or 15, respectively, and
optionally (ii) a primer pair consisting of a forward primer oligonucleotide comprising a sequence that is substantially identical to a stretch of contiguous nucleotides of SEQ ID NO: 32 or 35, and a reverse primer oligonucleotide comprising a sequence that is substantially identical to a stretch of contiguous nucleotides of SEQ ID NO: 33 or 34, respectively, wherein the primer pair is suitable for amplification of a sequence comprising one or more CpG dinucleotides of SEQ ID NO: 32 or 35, respectively.

In a fifth aspect, the present invention relates to the use of the method of the first aspect or of the kit of the fourth aspect for the detection of CRC, or for monitoring a subject having an increased risk of developing CRC, suspected of having CRC or that has had CRC.

In a sixth aspect, the present invention relates to a method of treating CRC in a subject in which the presence of CRC has been detected according to the method of the second aspect, or which is or has been monitored according to the method of the third aspect, with a treatment regimen suitable for treating CRC.

### LEGENDS TO THE FIGURES

FIG. 1: Map of preferred target regions. A: ANKRD13B, B: SEPTIN9. See Table 1 for an explanation of the SEQ ID NOs.

FIG. 2: Assay examples. The figures show primers (listed as A and B) and probe oligomer (oligo, C) either directly or—if matching the antisense strand—as reverse complement, their start and end position, and information for the strand they match on in relation to the genomic reference sequence of the amplified assay. Bisulfite specific base pairing (T on C for converted sense strand, G on A for converted antisense strand) to the genomic reference is displayed by ‘+’. Listed are all fout methylation specific assay examples A: ANKRD13BoB1, B: ANKRD13BoB2, C: SEPTIN90B1, D: SEPTIN90B2.

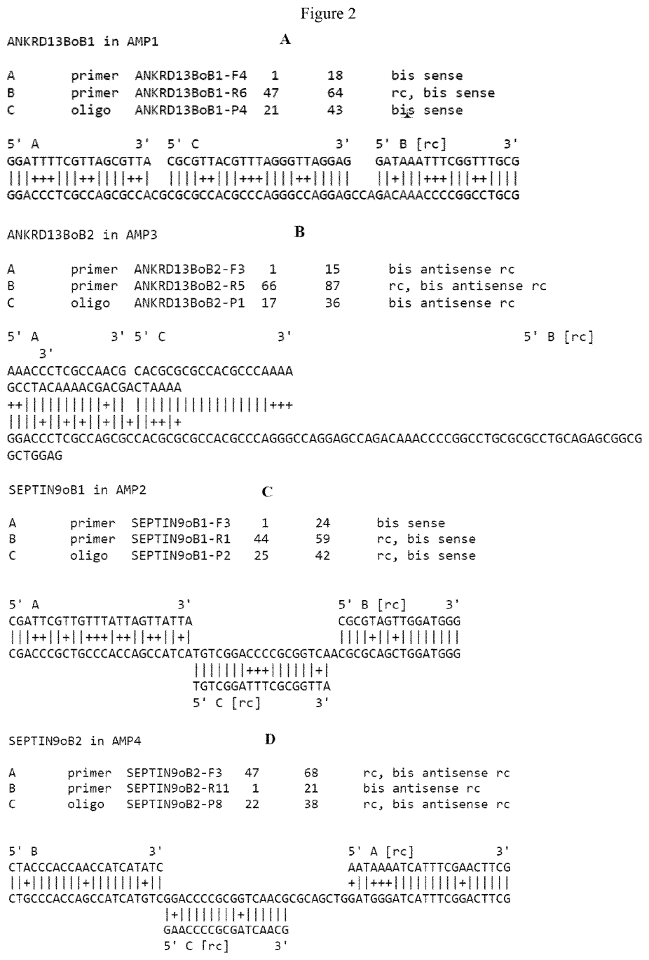

FIG. 3: Blood plasma derived Ct values of methylation specific Realtime PCR pentaplex assays. Each row visualizes data from an individual blood plasma sample, organized in blocks showing 15 CRCs and 16 NEDs (top to bottom). The first 12 columns show Cts of four methylation assay results in triplicate, sorted in 4 blocks for each assay, from left to right. ANKRD13b (A13b) assay on the bisulfite converted sense strand (bis1) in triplicate (1:3), triplicates of ANKRD13b assay on the reverse complement strand (bis2), Septin9 assay (S9) on the sense strand and Septin9 assay of the reverse complement strand. The four columns on the right display the minimum Ct for each of the assay as used for assessment method 1. White areas show results for assay replicates that showed no amplification curve. For CRCs, cancer stages from I to IV are displayed on the right side if available (NA means not available). Early (lower) Cts (darker grey) are correlated to more methylated template.

FIG. 4: Blood plasma derived Ct values of methylation specific Realtime PCR pentaplex assays. The data is organized as in FIG. 3, but display Ct values as numbers instead of a grey scale heat map. Areas without numbers represent results for assay replicates that showed no amplification curve.

FIG. 5: Receiver operating characteristic (ROC) curves from blood plasma samples based on assessment method 2 (A) and 3 (B). Numbers in the right lower corner show area under the curve (AUC) of ROC curves.

FIG. 6: Urine derived Ct values of methylation specific Realtime PCR pentaplex assays. Each row visualizes data from an individual urine sample, organized in blocks showing 5 CRCs and 6 NEDs (top to bottom). The first 12 columns show Cts of four methylation assay results in triplicate, sorted in 4 blocks for each assay, from left to right: ANKRD13b (A13b) assay on the bisulfite converted sense strand (bis1) in triplicate (1:3), triplicates of ANKRD13b assay on the reverse complement strand (bis2), Septin9 assay (S9) on the sense strand and Septin9 assay of the reverse complement strand. The four columns on the right display the minimum Ct for each of the assay. White areas show results for assay replicates that showed no amplification curve. Early (smaller) Cts (darker grey) are correlated to more methylated template.

FIG. 7: Urine derived Ct values of methylation specific Realtime PCR pentaplex assays. The data is organized as in FIG. 6, but display Ct values as numbers instead of a grey scale heat map. Areas without numbers represent results for assay replicates that showed no amplification curve.

FIG. 8: Receiver operating characteristic (ROC) curves from urine samples based on assessment method 2 (A) and 3 (B). Numbers in the right lower corner show area under the curve (AUC) of ROC curves.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturers' specifications, instructions etc.), whether supra or infra, is hereby incorporated by reference in its entirety.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

### Aspects of the Invention and Particular Embodiments Thereof

In a first aspect, the present invention relates to a method for detecting DNA methylation (i) at one or more CpG dinucleotides within genomic DNA having a sequence comprised in SEQ ID NO: 11 (mANKRD13B target region) and optionally (ii) at one or more CpG dinucleotides within genomic DNA having a sequence comprised in SEQ ID NO: 31 (mSEPTIN9 target region) in a biological sample of a subject. Preferably, the method is an in vitro method.

The genomic DNA may comprise DNA derived from colorectal cancer (CRC) cells. In a preferred embodiment, the genomic DNA, in particular the genomic DNA derived from CRC cells, is cell-free DNA (cell-free tumour DNA comprised in the total cell-free DNA of the sample) or more specifically circulating tumor DNA (ctDNA). The phrase “the genomic DNA may comprise DNA derived from colorectal cancer (CRC) cells” does, in a preferred embodiment, mean that the subject has an increased risk of CRC, is suspected of having CRC or has had CRC (i.e. has been treated to remove any detectable sign of CRC, but is suspected to relapse).

Depending on what the method of the first aspect is to be used for, the term “subject” may have different limitations. For example, if the method is to be used for detecting CRC or screening subjects for CRC, the subject is not known to have CRC, i.e. it may or may not have CRC. In this example, the subject preferably has an increased risk of developing or is suspected to have CRC, or has had CRC. “Increased risk” means that one or more risk factors for cancer generally or for CRC can be attributed to the subject, preferably as defined by the American Cancer Society for cancer generally or for CRC. Examples of risk factors for CRC are: heavy alcohol use (more than 3 or 4 alcohol units a day for men, or more than 2 or 3 alcohol units a day for women; an alcohol unit is defined as 10 ml (8 g) of pure alcohol), tobacco consumption (in particular smoking, but also including smokeless tobacco), being overweight (Body Mass Index (BMI) of 25 to 29.9) or obese (BMI of 30 or more), especially having a larger waistline, physical inactivity (exercise (sports) for less than 150, preferably 75 minutes per week beyond usual (non-sport) daily activities), diet rich in red meats (such as beef, pork, lamb or liver) and processed meats, age of 35 or older, preferably 40 or older, more preferably 45 or older, personal history of colorectal polyps, colorectal cancer and/or inflammatory bowel disease (e.g. ulcerative colitis or Crohn's disease), a familial history of colorectal cancer or adenomatous polyps (preferably first degree relative (parent, sibling or child), more preferably diagnosed at age 45 or younger and/or more than one first degree relative affected), having an inherited syndrome increased CRC risk such as preferably Lynch syndrome (hereditary non-polyposis colorectal cancer or HNPCC) or familial adenomatous polyposis (FAP), but also Peutz-Jeghers syndrome (PJS) or MYH-associated polyposis (MAP), racial and ethnic background with increased risk (e.g. African Americans or Ashkenazi Jews), and having type 2 diabetes. In one embodiment, the subject has a genetic, epigenetic or other known predisposition for CRC.

The term “colorectal cancer (CRC)”, also known as bowel cancer and colon cancer, is used in the broadest sense and refers to all cancers that start in the colon or in the rectum. It includes the subtypes adenocarcinoma (cancer starting in cells that make mucus to lubricate the inside of the colon and rectum), carcinoid tumor (cancer starting from the interstitial cells of Cajal (ICC) in the wall of the colon), lymphoma starting in the colon or rectum, and sarcoma starting in blood vessels, muscle layers, or other connective tissues in the wall of the colon and rectum. The most common and preferred CRC with regard to the invention is adenocarcinoma.

In one embodiment, the biological sample is a colon or rectum tissue sample or a liquid biopsy, preferably a blood sample, a blood-derived sample, a urine sample, a urine-derived sample, a saliva sample, or a saliva-derived sample. A sample derived from another sample comprises cell-free DNA from that sample. Preferred examples of a blood-derived sample are a plasma sample, a serum sample, or a sample derived from plasma or serum. A “colon or rectum tissue sample” is a tissue sample from any tissue in which CRC can occur. In one embodiment, if the subject has cancer, it is a CRC tissue sample.

The sequence a genomic region has is also referred to herein as the target region or target DNA and may be the sequence of the entire corresponding SEQ ID NO, or may be a sequence within having a length as specified below in the section “Definitions and further embodiments of the invention”.

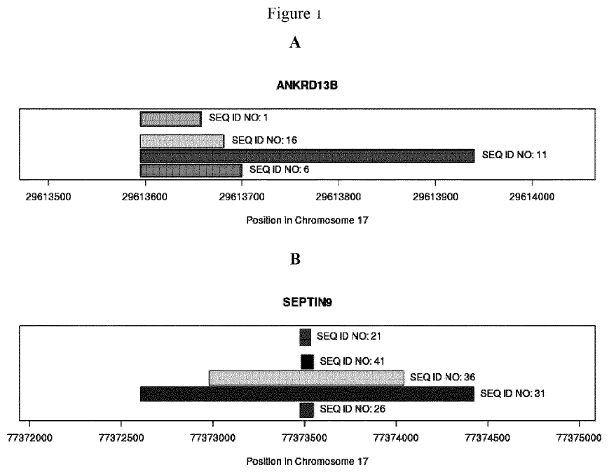

In this specification, the target DNAs are also referred to using the designations mANKRD13B and mSEPTIN9. In these, the first letter “m” means “methylation marker”, and the capital letters refer to the gene the target DNA resides in (the corresponding genomic region is provided in Table 1). When using these designations only without indicating specific SEQ ID NOs, it is referred to the SEQ ID NOs which correspond to the designation according to FIG. 1 and Table 1, with the order of preference indicated herein.

In a preferred embodiment, the genomic target DNA (the DNA region within which methylation is detected) comprises at least one CpG dinucleotide, preferably at least 2, 3, 4, or 5, most preferably at least 6 (e.g. at least 7, 8, 9, 10, 15 or 30) CpG dinucleotides. Generally, the methylation of at least one CpG dinucleotide comprised in the genomic DNA is detected, preferably of at least 2, 3, 4, or 5, most preferably at least 6 (e.g. at least 7, 8, 9, 10, 15 or 30) CpG dinucleotides. In one specifically preferred embodiment, DNA methylation is detected at 7 or more (preferably 8 or more) CpG dinucleotides within the mANKRD13B target region.

Preferred mANKRD13B target regions are as follows: the genomic DNA having a sequence comprised in SEQ ID NO: 11 has a sequence comprised in SEQ ID NO: 6, preferably in SEQ ID NO: 16, more preferably in SEQ ID NO: 1. Preferred mSEPTIN9 target regions are as follows: the genomic DNA having a sequence comprised in SEQ ID NO: 31 has a sequence comprised in SEQ ID NO: 36, preferably in SEQ ID NO: 26, more preferably in SEQ ID NO: 21 or SEQ ID NO: 41.

In one embodiment, the method of the first aspect comprises the steps of
(a) converting cytosine unmethylated in the 5-position to uracil or another base that does not hybridize to guanine in the genomic DNA of the biological sample; and
(b) detecting DNA methylation within the genomic DNA by detecting unconverted cytosine in the converted DNA of step (a).

A preferred way of carrying out the method comprises the steps of
(a) converting cytosine unmethylated in the 5-position to uracil or another base that does not hybridize to guanine in the genomic DNA;
(b) amplifying methylation-specifically a region of the converted DNA;
(c) detecting the presence or absence of DNA amplified in step (b);
wherein the presence or absence of amplified DNA indicates the presence or absence, respectively, of methylated genomic DNA.

In a preferred embodiment, step b) of amplifying comprises the use of oligonucleotides as comprised in the kit of the fourth aspect.

In a more preferred embodiment, the method comprises the steps of
(a) converting cytosine unmethylated in the 5-position to uracil or another base that does not hybridize to guanine in the genomic DNA of the biological sample; and
(b) detecting DNA methylation within the mANKRD13B target region by detecting unconverted cytosine in converted DNA according to SEQ ID NOs 12 and 15, and optionally detecting DNA methylation within the mSEPTIN9 target region by detecting unconverted cytosine in converted DNA according to SEQ ID NOs 32 and 35, preferably wherein methylation of at least one, more preferably each, of the one or more CpG dinucleotides within the mANKRD13B target region is detected on both the sense strand and the anti-sense strand, and optionally wherein methylation of at least one, more preferably each, of the one or more CpG dinucleotides within the mSEPTIN9 target region is detected on both the sense strand and the anti-sense strand. The inventors found that the detection of methylation on both strands, even if the same CpG sites are examined on both strands (i.e. the complementary CpG dinucleotides), increases accuracy of detection. This is particularly advantageous for the detection of CRC in liquid biopsy material e.g. blood plasma or urine—especially in early cancer stages-detection of methylated tumor DNA is limited by the availability of circulating tumor DNA (ctDNA), which may only be a few molecules. Under these circumstances, the examination of the same CpG sites on different strands can increase the chance of successfully detecting rare ctDNA by—in theory—examining twice as many molecules.

“Detecting DNA methylation” is also referred to herein as detecting or determining whether methylated DNA is present or absent. The term “presence of methylated genomic DNA” as used herein refers to a detectable amount of methylated genomic DNA, specifically detectable by PCR. In particular, it refers to the presence of a significant amount of methylated genomic DNA. A significant amount may be described as at least X molecules of the methylated target DNA per 100 μl of the sample used, preferably per 100 μl of blood, serum, plasma or urine. X may be as low as 1 and is usually a value between and including 1 and 50, in particular between and including 1 and 2, 3, 4, 5, 10, 15, 20, 25, 30 or 40. If X is low, the methylated targeted DNA can be detected by using a plurality of PCR reactions, e.g. between and including 2 and 10, or 2 and 5, for instance 3.

For determination whether there is sufficient DNA in the sample to contain a detectable amount, total DNA may be, but does not necessarily have to be quantified or checked for a minimum quantity. This can be done by determining the presence or a minimum amount of control DNA, preferably of a housekeeping gene such as ACTB (actin beta). This minimum amount can be defined as a maximum cycling threshold (Ct) of a real-time PCR, e.g. within a range from 30 to 34 (e.g. 32.1), preferably as would be obtained in an assay according to Example 1. In one embodiment, the minimum amount is, e.g. for bisulfite-converted genomic DNA derived from blood plasma, 0.1, 0.2, 0.25, 0.3, 0.4 or preferably 0.5 ng per PCR reaction (e.g. about 0.25 ng). This may correspond to approximately the same amounts of total genomic DNA per mL of blood plasma. The determination may also be made by comparison to a standard, for example a standard comprising genomic DNA and therein a certain amount of substantially fully methylated DNA, e.g. the equivalence of X genomes, wherein X is as above. Detecting the presence of methylated DNA may comprise such determination whether there is sufficient DNA in the sample, preferably comprising determining the presence or a minimum amount of control DNA. The determination whether a significant amount of methylated DNA is present or absent is not necessarily a fully quantitative determination, but is preferably semi-quantitative, e.g. as demonstrated in Example 1.

In an alternative embodiment, the determination of the presence of a significant amount may comprise a relative comparison to a control, wherein an amount higher than in the control is deemed a significant amount. The control would be a sample of the same sample type (e.g. plasma or urine) and from a subject who does not have CRC and preferably does not have any cancer, or the mean or average level of methylated target DNA of samples of the same sample type (e.g. plasma or urine) and from subjects who do not have CRC and preferably do not have any cancer.

In a preferred embodiment of detecting the presence of a significant amount of methylated genomic DNA, the method is carried out using a plurality of reactions for each target region and optionally each strand (sense and anti-sense) thereof (each reaction using a portion of the same sample), or “repetitions” or “replicates”, for example using duplicates or triplicates (or more). The results (e.g. real-time PCR Ct values) of the individual reactions can be aggregated for example by using mean, median, maximum or—preferably-minimum values over repetitions for each target region and optionally each strand (sense and anti-sense) of each target region. Aggregated results of the sense and anti-sense strand and optionally also of the target regions can be further combined to a single value, for example to a sum, e.g. after multiplying each aggregated result with predefined weights (for example as trained by logistic regression using a training sample set). The aggregated results or the single value (combination of aggregated results) can be used as such and set into relation to a scale, or it can be used to determine a probability that methylated genomic DNA is present, or it can be used to classify the sample as positive or negative for methylated genomic DNA based on a pre-defined threshold that can be optimized for sensitivity/specificity based on training data. As an alternative, each result (e.g. real-time PCR Ct values) of the individual reactions is “binarized”, i.e. classified as positive or negative (1 or 0), e.g. based on a predefined threshold. For semi-quantitative determination, the plurality of binarized results obtained can be combined, e.g. as a sum. For example, a real-time PCR for detecting methylation in both target regions (×2), both on the sense and the anti-sense strand (×2) in triplicates (×3) yields 12 (2×2×3) binary results which can be added to give a score of 0 to 12. This score can be used to classify the sample as positive or negative for methylated genomic DNA based on a pre-defined threshold (any one of 1, 2, 3, 4, 5, 6, 7, 8, 9, 10, 11 and 12, preferably any one of 4, 5, 6, 7, 8, 9, 10, 11 and 12 or any one of 8, 9, 10, 11 and 12).

Accordingly, a significant amount can be deemed to be present if, for example, if a certain number of PCR reactions reaches a pre-defined Ct threshold (e.g. 45, 44, 43, 42, 41, 40, 39, 38, 37, 36 or 35, wherein lower Cts are preferred). The certain number may for example be at least 50%, 60%, 70%, 80% or 90% of the total number of PCR reactions. Another example of a determination is that a significant amount can be deemed to be present if all replicates of at least one marker assay reaches the pre-defined Ct threshold, or one or more than one (but not all) of the replicates of at least two marker assays reach the pre-defined Ct threshold. Many alternative ways of determining the presence of whether a significant amount of methylated DNA are conceivable and the present invention is not limited to one or more particular ways, provided they allow for discriminating between samples from subjects with CRC and samples from subjects without CRC, preferably without any cancer.

Thus, in a preferred embodiment of the method of the first aspect, the detecting of the DNA methylation comprises determining the amount of methylated genomic DNA, preferably semi-quantitatively, e.g. by determining the number of amplification cycles necessary to detect an amplificate (real-time PCR Ct value), and/or the number of positive signals (i.e. amplificates detected) in a plurality of PCR reactions (optionally including PCR reactions for both the sense and the anti-sense strand, as described herein).

The amplification is preferably performed by MSP (methylation-specific PCR) (i.e. an amplicon is produced depending on whether one or more CpG sites are converted or not) using at least one primer that is methylation-specific (and specific to bisulfite-converted DNA). Alternatively, primers may be methylation-unspecific, but specific to bisulfite-converted DNA (i.e. hybridize only to converted DNA by covering at least one converted C). In this alternative case, methylation-specificity is achieved by using methylation-specific blocker oligonucleotides, which hybridize specifically to converted or non-converted CpG sites and thereby terminate the PCR polymerization. In a preferred embodiment, the step of amplifying comprises a PCR, preferably a real-time PCR, wherein the converted DNA is amplified methylation-specifically by using at least one methylation-specific primer in a pair of primers. In specific embodiments, the region of the converted DNA is amplified methylation-specifically by using a pair of methylation-specific primers. The presence of amplified DNA is preferably detected by using an oligonucleotide probe, more preferably a methylation-specific oligonucleotide probe. In one embodiment, each amplificate generated with a pair of primers is detected with one or more (preferably one) of such probes, i.e. a primer pair has corresponding probes. Additional characteristics of the primers and probes are defined further below.

In a preferred embodiment, the PCR is a multiplex real-time PCR comprising at least one of (1) and (2) and at least one of (3) to (5) of
(1) primers suitable for amplifying DNA within SEQ ID NO: 12,
(2) primers suitable for amplifying DNA within SEQ ID NO: 15,
(3) primers suitable for amplifying DNA within SEQ ID NO: 32, and
(4) primers suitable for amplifying DNA within SEQ ID NO: 35, and
(5) methylation-unspecific primers suitable for amplifying control DNA,
wherein the primers of (1) to (4) preferably are methylation-specific primers. For example, it comprises:
   (1) and/or (2), and (5);
   (1) and (2), (3) or (4), and (5); or
   (1) or (2), (3) and (4), and (5).

More preferably, the multiplex real-time PCR comprises
(1) primers suitable for amplifying DNA within SEQ ID NO: 12,
(2) primers suitable for amplifying DNA within SEQ ID NO: 15,
(3) primers suitable for amplifying DNA within SEQ ID NO: 32, and
(4) primers suitable for amplifying DNA within SEQ ID NO: 35, and
optionally (5) methylation-unspecific primers suitable for amplifying control DNA,
wherein the primers of (1) to (4) preferably are methylation-specific primers.

The control DNA is preferably DNA of a housekeeping gene present in the sample, preferably ACTB (actin beta) DNA.

Definitions and embodiments described below, in particular under the header ‘Definitions and further embodiments of the invention’ apply to the method of the first aspect.

In a second aspect, the present invention relates to a method for detecting colorectal cancer (CRC) in a subject, comprising detecting DNA methylation according to the first aspect, wherein
(1), if the mANKRD13B target region and not the mSEPTIN9 target region is examined, the presence of detected methylated genomic DNA indicates the presence of CRC and the absence of detected methylated genomic DNA indicates the absence of CRC, and
(2), if both the mANKRD13B target region and the mSEPTIN9 target region are examined, the presence of detected methylated genomic DNA of at least one of the target regions indicates the presence of CRC.

In one embodiment of (2), if both the mANKRD13B target region and the mSEPTIN9 target region are examined, the presence of detected methylated genomic DNA of one or both (wherein one is sufficient and both are preferred) of the target regions indicates the presence of CRC.

In a preferred embodiment, the absence of detected methylated genomic DNA of both target regions indicates the absence of CRC.

The cancer may be of any subtype and stage as defined below, i.e. the presence or absence of any subtype and/or stage can be detected.

In a particular embodiment, the method of the second aspect further comprises confirming CRC by using one or more further means for detecting CRC. The further means may be a cancer marker (or “biomarker”) or a conventional (non-marker) detection means. The cancer marker can for example be a DNA methylation marker, a mutation marker (e.g. SNP), an antigen marker, a protein marker, a miRNA marker, a cancer specific metabolite, or an expression marker (e.g. RNA or protein expression). The conventional means can for example be a biopsy (e.g. visual biopsy examination with or without staining methods for example for protein or expression markers), an imaging technique (e.g. X-ray imaging, CT scan, nuclear imaging such as PET and SPECT, ultrasound, magnetic resonance imaging (MRI), thermography, endoscopy, digital mammography, colonoscopy or virtual colonoscopy, laparoscopy, angiogram, bone scan or sentinel node mapping for breast cancer) or a physical, e.g. tactile examination. It is preferred that it is a biopsy or other means that removes and examines a solid tissue sample of the subject from colorectal tissue. Generally, the further means is suitable for detecting CRC. Preferred examples are colonoscopy, blood test for anemia and/or carcinoembryonic antigen (CEA), CR scan, MRI, ultrasound, X-ray, and PET scan.

As indicated above with respect to the first aspect, the “presence/absence of detected methylated genomic DNA” preferably means, here and below, the “presence/absence of a significant amount of methylated genomic DNA”.

Definitions given and embodiments described with respect to the first aspect apply also to the second aspect, in as far as they are applicable. Also, definitions and embodiments described below, in particular under the header ‘Definitions and further embodiments of the invention’ apply to the method of the second aspect.

In a third aspect, the present invention relates to a method for monitoring a subject suspected of having CRC, having an increased risk of developing CRC, or who has had CRC, comprising detecting DNA methylation according to the first aspect repeatedly, wherein
(1), if the mANKRD13B target region and not the mSEPTIN9 target region is examined, the presence of detected methylated genomic DNA indicates the presence of CRC and the absence of detected methylated genomic DNA indicates the absence of CRC, and
(2), if both the mANKRD13B target region and the mSEPTIN9 target region are examined, the presence of detected methylated genomic DNA of at least one of the target regions indicates the presence of CRC.

Definitions given and embodiments described with respect to the first and second aspect apply also to the third aspect, in as far as they are applicable. Also, definitions and embodiments described below, in particular under the header ‘Definitions and further embodiments of the invention’ apply to the oligonucleotide of the third aspect.

In a fourth aspect, the present invention relates to a kit comprising (i) a primer pair consisting of a forward primer oligonucleotide comprising a sequence that is substantially identical to a stretch of contiguous nucleotides of SEQ ID NO: 12 or 15, and a reverse primer oligonucleotide comprising a sequence that is substantially identical to a stretch of contiguous nucleotides of SEQ ID NO: 13 or 14, respectively, wherein the primer pair is suitable for amplification of a sequence comprising one or more CpG dinucleotides of SEQ ID NO: 12 or 15, respectively, and
optionally (ii) a primer pair consisting of a forward primer oligonucleotide comprising a sequence that is substantially identical to a stretch of contiguous nucleotides of SEQ ID NO: 32 or 35, and a reverse primer oligonucleotide comprising a sequence that is substantially identical to a stretch of contiguous nucleotides of SEQ ID NO: 33 or 34, respectively, wherein the primer pair is suitable for amplification of a sequence comprising one or more CpG dinucleotides of SEQ ID NO: 32 or 35, respectively.

In a preferred embodiment, the kit comprises:
(1) a first primer pair consisting of a first forward primer oligonucleotide comprising a sequence that is substantially identical to a stretch of contiguous nucleotides of SEQ ID NO: 12, and a first reverse primer oligonucleotide comprising a sequence that is substantially identical to a stretch of contiguous nucleotides of SEQ ID NO: 13, wherein the primer pair is suitable for amplification of a sequence comprising one or more CpG dinucleotides of SEQ ID NO: 12, and/or (preferably and)
(2) a second primer pair consisting of a second forward primer oligonucleotide comprising a sequence that is substantially identical to a stretch of contiguous nucleotides of SEQ ID NO: 15, and a second reverse primer oligonucleotide comprising a sequence that is substantially identical to a stretch of contiguous nucleotides of SEQ ID NO: 14, wherein the primer pair is suitable for amplification of a sequence comprising one or more CpG dinucleotides of SEQ ID NO: 15.

In a more preferred embodiment, the kit further comprises
(3) a third primer pair consisting of a third forward primer oligonucleotide comprising a sequence that is substantially identical to a stretch of contiguous nucleotides of SEQ ID NO: 32, and a third reverse primer oligonucleotide comprising a sequence that is substantially identical to a stretch of contiguous nucleotides of SEQ ID NO: 33, wherein the primer pair is suitable for amplification of a sequence comprising one or more CpG dinucleotides of SEQ ID NO: 32, and/or (preferably and)
(4) a fourth primer pair consisting of a fourth forward primer oligonucleotide comprising a sequence that is substantially identical to a stretch of contiguous nucleotides of SEQ ID NO: 35, and a fourth reverse primer oligonucleotide comprising a sequence that is substantially identical to a stretch of contiguous nucleotides of SEQ ID NO: 34, wherein the primer pair is suitable for amplification of a sequence comprising one or more CpG dinucleotides of SEQ ID NO: 35, and
optionally (5) a fifth primer pair consisting of a methylation-unspecific fifth forward primer oligonucleotide and a methylation-unspecific fifth reverse primer oligonucleotide suitable for amplifying control DNA.

In another embodiment, the kit comprises at least one of (1) and (2) and at least one of (3) to (5) of the above. For example, it comprises:
(1) and/or (2), and (5);
(1) and (2), (3) or (4), and (5); or
(1) or (2), (3) and (4), and (5).

It is preferred that the primer pair suitable for amplification of a sequence comprising one or more CpG dinucleotides of SEQ ID NO: 12 is suitable for amplification of a sequence comprising 7 or more (preferably 8 or more) CpG dinucleotides of SEQ ID NO: 12, and/or the primer pair suitable for amplification of a sequence comprising one or more CpG dinucleotides of SEQ ID NO: 15 is suitable for amplification of a sequence comprising 7 or more (preferably 8 or more) CpG dinucleotides of SEQ ID NO: 15.

Preferred mANKRD13B primers are as follows: a primer oligonucleotide comprising a sequence that is substantially identical to a stretch of contiguous nucleotides of SEQ ID NO: 12, 13, 14 or 15 comprises a sequence that is substantially identical to a stretch of contiguous nucleotides of SEQ ID NO: 7, 8, 9 or 10, respectively; preferably SEQ ID NO: 17, 18, 19 or 20, respectively; and more preferably SEQ ID NO: 2, 3, 4 or 5, respectively. Preferred mSEPTIN9 primers are as follows: a primer oligonucleotide comprising a sequence that is substantially identical to a stretch of contiguous nucleotides of SEQ ID NO: 32, 33, 34 or 35 comprises a sequence that is substantially identical to a stretch of contiguous nucleotides of SEQ ID NO: 37, 38, 39 or 40, respectively; preferably SEQ ID NO: 27, 28, 29 or 30, respectively; and more preferably SEQ ID NO: 22, 23, 24 or 25, respectively, or SEQ ID NO: 42, 43, 44 or 45, respectively.

Generally, it is preferred that the kit comprises one or more (preferably one) probe oligonucleotides for each primer pair the kit comprises (“corresponding primer pair”). The probes are capable of binding to a strand of the amplificate the primer pair is suitable to generate in a PCR, preferably to a site between the primer binding sites or overlapping with one or both of the primer binding sites of the corresponding primer pair. Accordingly, a probe oligonucleotide is substantially identical to a stretch of contiguous nucleotides of the SEQ ID containing the sequence (or a sequence substantially identical thereto) of one of the primers of the corresponding primer pair.

Preferably, a primer or probe oligonucleotide comprises at least 1, 2 or 3 CpG dinucleotides, which makes it a methylation-specific oligonucleotide. This methylation-specific oligonucleotide is also specific for bisulfite-converted DNA, since it comprises at least one nucleotide derived from conversion of a C not in a CpG context (e.g. of a CpC, CpA or CpT dinucleotide) in SEQ ID NO: 11 (alternatively SEQ ID NO: 31 or a sub-sequence of either as recited above) or its complement into a T.

A probe oligonucleotide preferably has one or more modifications selected from the group consisting of a detectable label and a quencher, and/or a length of 5-40 nucleotides. Further, a probe is preferably a methylation-specific oligonucleotide. A primer preferably has a length of 10-40 nucleotides.

Generally, an oligonucleotide comprising a certain sequence preferably is an oligonucleotide having (or consisting of) that sequence.

Definitions given and embodiments described with respect to the first, second and third aspect apply also to the fourth aspect, in as far as they are applicable. Also, definitions and embodiments described below, in particular under the header ‘Definitions and further embodiments of the invention’ apply to the kit of the fourth aspect.

In a fifth aspect, the present invention relates to the use of the method of the first aspect, or of the kit of the fourth aspect for the detection of CRC, or for monitoring a subject having an increased risk of developing CRC, suspected of having CRC or that has had CRC. Preferably, the use is an in vitro use.

Definitions given and embodiments described with respect to the first, second, third and fourth aspect apply also to the fifth aspect, in as far as they are applicable. Also, definitions and embodiments described below, in particular under the header ‘Definitions and further embodiments of the invention’ apply to the use of the fifth aspect.

In a sixth aspect, the present invention relates to a method of treating CRC in a subject in which the presence of CRC has been detected according to the method of the second aspect, or which is or has been monitored according to the method of the third aspect, with a treatment regimen suitable for treating CRC. It also relates to a method of treatment, comprising the method of the first or the second aspect, or the use of the fifth aspect and a step of treating CRC of a subject for which the DNA methylation is detected in its biological sample. It also relates to a method of treatment, comprising treating CRC in a subject for which DNA methylation has been detected according to the method of the first aspect, or the use of the fifth aspect.

The present invention also relates to a method comprising the method of the first, the second or the third aspect and subsequently referring a subject for such a cancer treatment. Definitions and further embodiments for the cancer treatment regimen are given below.

Definitions given and embodiments described with respect to the first, second, third, fourth and fifth aspect apply also to the sixth aspect, in as far as they are applicable. Also, definitions and embodiments described below, in particular under the header ‘Definitions and further embodiments of the invention’ apply to the method of the sixth aspect.

### Definitions and Further Embodiments of the Invention

The specification uses a variety of terms and phrases, which have certain meanings as defined below. Preferred meanings are to be construed as preferred embodiments of the aspects of the invention described herein. As such, they and also further embodiments described in the following can be combined with any embodiment of the aspects of the invention and in particular any preferred embodiment of the aspects of the invention described above.

The term “detecting DNA methylation” as used herein refers to at least qualitatively analysing for the presence or absence of methylated target DNA. Methylation is preferably determined at 1 or more, 2 or more, 3 or more, 4 or more, 5 or more, 6 or more, 7 or more, 8 or more, 9 or more, 10 or more, 15 or more or 30 or more CpG sites of the target DNA. Usually, the CpG sites analysed are co-methylated in cancer, such that also CpG sites of neighbouring DNA are methylated and can be analysed in addition or instead. “At least qualitatively” means that also a quantitative determination of methylated target DNA, if present, can be performed. Such a “determining the amount” can be performed as described herein.

The presence or absence of amplified DNA can be detected by any means known in the art, e.g. autoradiography, silver staining or ethidium bromide staining. Preferably, the presence or absence of DNA amplified in step (b) is detected by real-time PCR or by sequencing the amplified DNA.

In a real-time PCR, the presence of DNA amplified in step (b) is preferably detected by using a methylation-specific oligonucleotide which is a probe. The DNA is preferably amplified methylation-specifically using methylation-specific primers or alternatively a methylation-specific blocker with methylation-unspecific primers, the former being preferred.

A detection by sequencing is preferably a detection by Next Generation Sequencing. Therein, the converted (e.g. bisulfite converted) methylated target DNA of the sample can be amplified bisulfite-specifically and can but must not necessarily be amplified methylation-specifically. Then, the amplified sequences are sequenced and the presence of methylated template is deduced from the presence of sequences or sequence reads derived from the amplified converted target DNA.

Furthermore, the absolute or relative amount of methylated target DNA may be determined by sequencing, preferably Next Generation Sequencing. Therein, the converted (e.g. bisulfite converted) target DNA can be amplified either methylation specifically, i.e. the target DNA is amplified only if it is methylated, or methylation-unspecifically, i.e. the target DNA is amplified whether or not it is methylated (in other words whether or not cytosines of the CpG sites are converted). This can be achieved by bisulfite-specific primers which either are or are not methylation-specific, respectively. Then, the amplified sequences are sequenced and subsequently counted. For methylation-specific amplification product, the number of determined sequences can be used to estimate the total number of methylated target molecules. For methylation-unspecific amplification product, the ratio of sequences derived from converted methylated DNA (identified in the sequences by CpG sites) and sequences derived from converted unmethylated DNA is calculated, resulting in a (relative) amount of methylated target DNA.

The term “Next Generation Sequencing” (NGS, also known as 2nd or 3rd generation sequencing) refers to a sequencing the bases of a small fragment of DNA are sequentially identified from signals emitted as each fragment is re-synthesized from a DNA template strand. NGS extends this process across millions of reactions in a massively parallel fashion, rather than being limited to a single or a few DNA fragments. This advance enables rapid sequencing of the amplified DNA, with the latest instruments capable of producing hundreds of gigabases of data in a single sequencing run. See, e.g., Shendure and Ji, Nature Biotechnology 26, 1135-1145 (2008) or Mardis, Annu Rev Genomics Hum Genet. 2008; 9:387-402. Suitably NGS platforms are available commercially, e.g. the Roche 454 platform, the Roche 454 Junior platform, the Illumina HiSeq or MiSeq platforms, or the Life Technologies SOLID 5500 or Ion Torrent platforms.

Substantially fully methylated genomic DNA preferably is DNA, particularly genomic DNA, which has all or substantially all CpG sites methylated. “Substantially all” in this respect means at least 95%, 96%, 97%, 98%, 99%, 99.5% or 99.9%. In a preferred embodiment, the methylation of all or substantially all CpG sites is achieved by treating the DNA with a CpG methyltransferase in a manner that provides for the methylation of all or substantially all CpG sites.

The term “methylated” or “hypermethylated” as used herein refers to a biochemical process involving the addition of a methyl group to the cytosine or adenine DNA nucleotides. DNA methylation at the 5 position of cytosine, especially in promoter regions, can have the effect of reducing gene expression and has been found in every vertebrate examined. In adult non-gamete cells, DNA methylation typically occurs in a CpG site. The term “CpG site” or “CpG dinucleotide”, as used herein, refers to regions of DNA where a cytosine nucleotide occurs next to a guanine nucleotide in the linear sequence of bases along its length. “CpG” is shorthand for “C-phosphate-G”, that is cytosine and guanine separated by only one phosphate; phosphate links any two nucleosides together in DNA. The “CpG” notation is used to distinguish this linear sequence from the CG base-pairing of cytosine and guanine. Cytosines in CpG dinucleotides can be methylated to form 5-methylcytosine. The term “CpG site” or “CpG site of genomic DNA” is also used with respect to the site of a former (unmethylated) CpG site in DNA in which the unmethylated C of the CpG site was converted to another as described herein (e.g. by bisulfite to uracil). The application provides the genomic sequence of each relevant DNA region as well as the bisulfite converted sequences of each converted strand. CpG sites referred to are always the CpG sites of the genomic sequence, even if the converted sequence does no longer contain these CpG sites due to the conversion. Specifically, methylation in the context of the present invention means hypermethylation. The term “hypermethylation” refers to an aberrant methylation pattern or status (i.e. the presence or absence of methylation of one or more nucleotides), wherein one or more nucleotides, preferably C(s) of a CpG site(s), are methylated compared to the same genomic DNA from a non-cancer cell of the subject or a subject not suffering or having suffered from the cancer the subject is treated for, preferably any cancer (healthy control). In particular, it refers to an increased presence of 5-mCyt at one or a plurality of CpG dinucleotides within a DNA sequence of a test DNA sample, relative to the amount of 5-mCyt found at corresponding CpG dinucleotides within a healthy control DNA sample. Hypermethylation as a methylation status/pattern can be determined at one or more CpG site(s). If more than one CpG site is used, hypermethylation can be determined at each site separately or as an average of the CpG sites taken together. Alternatively, all assessed CpG sites must be methylated such that the requirement hypermethylation is fulfilled.

Methylation is detected in particular in a region of the DNA according to the SEQ ID referred to (the “target DNA”). The term “target DNA” as used herein refers to a genomic nucleotide sequence at a specific chromosomal location. In the context of the present invention, it is typically a genetic marker that is known to be methylated in the state of disease (for example in cancer cells vs. non-cancer cells). A genetic marker can be a coding or non-coding region of genomic DNA.

The term “region of the target DNA” or “region of the converted DNA” as used herein refers to a part of the target DNA which is to be analysed. Generally, the region is at least 40, 50, 60, 70, 80, 90, 100, or 150 base pairs (bp) long and/or not longer than 150, 200, 300, 400, 500, or 1000 bp (e.g. 50-200, preferably 70-150 bp). In particular, it is a region comprising at least 3, 4, 5, 6, 7, 8, 9, 10, 11, 12, 13, 14 or 15 CpG sites of the genomic DNA. Preferably, at least 3, 4, 5, 6, 7, 8, 9, 10, 11, 12, 13, 14 or 15 (but not necessarily all, in particular CpG sites covered by a spacer or methylation-unspecific mismatch, e.g. in a primer) of these CpG sites are methylated in the target DNA.

The target DNAs of the invention are given in FIG. 1 and Table 1.

The term “sample” as used herein refers to biological material obtained from a subject and comprises genomic DNA from all chromosomes, preferably genomic DNA covering the whole genome. The sample comprises, if a subject has cancer, cell-free genomic DNA (including the target DNA) from cancer cells, preferably circulating genomic DNA from cancer cells.

The term “sample comprising cell-free DNA from blood” as used herein refers to a body fluid sample comprising cell-free DNA from blood. While in a preferred embodiment this sample is blood, the term also comprises other body fluids. For example, urine comprises cell-free DNA from blood. The term “sample derived from a sample comprising cell-free DNA from blood” as used herein refers to any sample that is derived by in vitro processing. For example, if the sample is blood, it is preferred that the sample derived therefore is plasma or serum.

The term “cell-free DNA” as used herein or its synonyms “cfDNA”, “extracellular DNA”, “circulating DNA” and “free circulating DNA” refers to DNA that is not comprised within an intact cell in the respective body fluid which is the sample or from which the sample is derived, but which is freely circulating in the body liquid sample. Cell-free DNA usually is genomic DNA that is fragmented as described below. The term “circulating tumor DNA” as used herein or its abbreviation “ctDNA”, refers to cell-free DNA, which originated from solid tumor tissue, metastases or circulating tumor cells and which usually comprises the target DNA.

Typically, in samples comprising the target DNA, especially extracellular target DNA, from cancer cells, there is also target DNA from non-cancer cells which is not methylated contrary to the target DNA from cancer cells. Usually, said target DNA from non-cancer cells exceeds the amount from diseased cells by at least 10-fold, at least 100-fold, at least 1,000-fold or at least 10,000-fold. Generally, the genomic DNA comprised in the sample is at least partially fragmented. “At least partially fragmented” means that at least the extracellular DNA, in particular at least the extracellular target DNA, from cancer cells, is fragmented. The term “fragmented genomic DNA” refers to pieces of DNA of the genome of a cell, in particular a cancer cell, that are the result of a partial physical, chemical and/or biological break-up of the lengthy DNA into discrete fragments of shorter length. Particularly, “fragmented” means fragmentation of at least some of the genomic DNA, preferably the target DNA, into fragments shorter than 1,500 bp, 1,300 bp, 1,100 bp, 1,000 bp, 900 bp, 800 bp, 700 bp, 600 bp, 500 bp, 400 bp, 300 bp, 200 bp or 100 bp. “At least some” in this respect means at least 5%, 10%, 20%, 30%, 40%, 50% or 75%.

The term “genomic DNA” as used herein refers to chromosomal DNA and is used to distinguish from coding DNA. As such, it includes exons, introns as well as regulatory sequences, in particular promoters, belonging to a gene.

The phrase “converting, in DNA, cytosine unmethylated in the 5-position to uracil or another base that does not hybridize to guanine” as used herein refers to a process of chemically treating the DNA in such a way that all or substantially all of the unmethylated cytosine bases are converted to uracil bases, or another base which is dissimilar to cytosine in terms of base pairing behaviour, while the 5-methylcytosine bases remain unchanged. The conversion of unmethylated, but not methylated, cytosine bases within the DNA sample is conducted with a converting agent. The term “converting agent” as used herein relates to a reagent capable of converting an unmethylated cytosine to uracil or to another base that is detectably dissimilar to cytosine in terms of hybridization properties. The converting agent is preferably a bisulfite such as disulfite, or hydrogen sulfite. The reaction is performed according to standard procedures (Frommer et al., 1992, Proc Natl Acad Sci USA 89:1827-31; Olek, 1996, Nucleic Acids Res 24:5064-6; EP 1394172). It is also possible to conduct the conversion enzymatically, e.g by use of methylation specific cytidine deaminases. Most preferably, the converting agent is sodium bisulfite or bisulfite.

The term “annealing”, when used with respect to an oligonucleotide, is to be understood as a bond of an oligonucleotide to an at least substantially complementary sequence along the lines of the Watson-Crick base pairings in the sample DNA, forming a duplex structure, under moderate or stringent hybridization conditions. When it is used with respect to a single nucleotide or base, it refers to the binding according to Watson-Crick base pairings, e.g. C-G, A-T and A-U. Stringent hybridization conditions involve hybridizing at 68° C. in 5×SSC/5×Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderate conditions involve washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

“Substantially complementary” means that an oligonucleotide does not need to reflect the exact sequence of the template and can comprise mismatches and/or spacers as defined herein. “Substantially identical” means that an oligonucleotide does not need to be 100% identical to a reference sequence but can comprise mismatches and/or spacers as defined herein. It is preferred that a substantially complementary or identical oligonucleotide comprises up to 1 to 3, i.e. 1, 2 or 3 mismatches and/or spacers, preferably up to one mismatch or spacer per oligonucleotide, such that the intended annealing does not fail due to the mismatches and/or spacers. To enable annealing despite mismatches and/or spacers, it is preferred that an oligonucleotide does not comprise more than 1 mismatch per 10 nucleotides (rounded up if the first decimal is 5 or higher, otherwise rounded down) of the oligonucleotide.

The term “oligonucleotide” as used herein refers to a linear oligomer of 5 to 50 ribonucleotides or preferably deoxyribonucleotides. Preferably, it has the structure of a single-stranded DNA fragment.

The term “primer oligonucleotide” as used herein refers to a single-stranded oligonucleotide sequence substantially complementary to a nucleic acid sequence sought to be copied (the template) and serves as a starting point for synthesis of a primer extension product. Preferably, a primer oligonucleotide is 10 to 40 nucleotides, more preferably 15-30 nucleotides and most preferably 19 to 25 nucleotides in length.

The term “blocker” as used herein refers to a molecule which binds in a methylation-specific manner to a single-strand of DNA (i.e. it is specific for either the converted methylated or preferably for the converted unmethylated DNA or the amplified DNA derived from it) and prevents amplification of the DNA by binding to it, for example by preventing a primer to bind or by preventing primer extension where it binds. Non-limiting examples for blockers are sequence and/or methylation specific antibodies (blocking e.g. primer binding or the polymerase) and in particular blocker oligonucleotides.

A “blocker oligonucleotide” may be a blocker that prevents the extension of the primer located upstream of the blocker oligonucleotide. It comprises nucleosides/nucleotides having a backbone resistant to the 5′ nuclease activity of the polymerase. This may be achieved, for example, by comprising peptide nucleic acid (PNA), locked nucleic acid (LNA), Morpholino, glycol nucleic acid (GNA), threose nucleic acid (TNA), bridged nucleic acids (BNA), N3′-P5′ phosphoramidate (NP) oligomers, minor groove binder-linked-oligonucleotides (MGB-linked oligonucleotides), phosphorothioate (PS) oligomers, CrC₄alkylphosphonate oligomers, phosphoramidates, β-phosphodiester oligonucleotides, a-phosphodiester oligonucleotides or a combination thereof. Alternatively, it may be a non-extendable oligonucleotide with a binding site on the DNA single-strand that overlaps with the binding site of a primer oligonucleotide. When the blocker is bound, the primer cannot bind and therefore the amplicon is not generated. When the blocker is not bound, the primer-binding site is accessible and the amplicon is generated. For such an overlapping blocker, it is preferable that the affinity of the blocker is higher than the affinity of the primer for the DNA. A blocker oligonucleotide is typically 15 to 50, preferably 20 to 40 and more preferably 25 to 35 nucleotides long. A blocker oligonucleotide cannot by itself act as a primer (i.e. cannot be extended by a polymerase) due to a non-extensible 3′ end.

The term “probe oligonucleotide” or “probe” as used herein refers to an oligonucleotide that is used to detect an amplicon by annealing to one strand of the amplicon, usually not where any of the primer oligonucleotides binds (i.e. not to a sequence segment of the one strand which overlaps with a sequence segment a primer oligonucleotide anneals to). Preferably it anneals without a mismatch or spacer, in other words it is preferably complementary to one strand of the amplicon. A probe oligonucleotide is preferably 5-40 nucleotides, more preferably 10 to 25 and most preferably 20 to 25 nucleotides long. Usually, the probe is linked, preferably covalently linked, to at least one detectable label which allows detection of the amplicon and/or at least one quencher which allows quenching the signal of a (preferably the) detectable label. The term “detectable label” as used herein does not exhibit any particular limitation. The detectable label may be selected from the group consisting of radioactive labels, luminescent labels, fluorescent dyes, compounds having an enzymatic activity, magnetic labels, antigens, and compounds having a high binding affinity for a detectable label. For example, fluorescent dyes linked to a probe may serve as a detection label, e.g. in a real-time PCR. Suitable radioactive markers are P-32, S-35, I-125, and H-3, suitable luminescent markers are chemiluminescent compounds, preferably luminol, and suitable fluorescent markers are preferably dansyl chloride, fluorcein-5-isothiocyanate, and 4-fluor-7-nitrobenz-2-aza-1,3 diazole, in particular 6-Carboxyfluorescein (FAM), 6-Hexachlorofluorescein (HEX), 5 (6)-Carboxytetramethylrhodamine (TAMRA), 5(6)-Carboxy-X-Rhodamine (ROX), Cyanin-5-Fluorophor (Cy5) and derivates thereof, suitable enzyme markers are horseradish peroxidase, alkaline phosphatase, a-galactosidase, acetylcholinesterase, or biotin. A probe may also be linked to a quencher. The term “quencher” as used herein refers to a molecule that deactivates or modulates the signal of a corresponding detectable label, e.g. by energy transfer, electron transfer, or by a chemical mechanism as defined by IUPAC (see compendium of chemical terminology 2^{nd} ed. 1997). In particular, the quencher modulates the light emission of a detectable label that is a fluorescent dye. In some cases, a quencher may itself be a fluorescent molecule that emits fluorescence at a characteristic wavelength distinct from the label whose fluorescence it is quenching. In other cases, the quencher does not itself fluoresce (i.e., the quencher is a “dark acceptor”). Such quenchers include, for example, dabcyl, methyl red, the QSY diarylrhodamine dyes, and the like.

The term “covering a CpG site” as used herein with respect to an oligonucleotide refers to the oligonucleotide annealing to a region of DNA comprising this CpG site, before or after conversion of the C of the CpG site (i.e. the CpG site of the corresponding genomic DNA when it is referred to a bisulfite converted sequence). The annealing may, with respect to the CpG site (or former CpG site if the C was converted), be methylation-specific or methylation-unspecific as described below.

The term “methylation-specific” as used herein refers generally to the dependency from the presence or absence of CpG methylation.

The term “methylation-specific” as used herein with respect to an oligonucleotide means that the oligonucleotide does or does not anneal to a single-strand of DNA (in which cytosine unmethylated in the 5-position has been converted to uracil or another base that does not hybridize to guanine, and where it comprises at least one CpG site before conversion) without a mismatch regarding the position of the C in the at least one CpG site, depending on whether the C of the at least one CpG sites was unmethylated or methylated prior to the conversion, i.e. on whether the C has been converted or not. The methylation-specificity can be either positive (the oligonucleotide anneals without said mismatch if the C was not converted) or negative (the oligonucleotide anneals without said mismatch if the C was converted). To prevent annealing of the oligonucleotide contrary to its specificity, it preferably covers at least 2, 3, 4, 5 or 6 and preferably 3 to 6 CpG sites before conversion.

The term “methylation-unspecific” as used herein refers generally to the independency from the presence or absence of CpG methylation. With respect to an oligonucleotide it means that the oligonucleotide does anneal to a single-strand of DNA (in which cytosine unmethylated in the 5-position has been converted to uracil or another base that does not hybridize to guanine, and where it may or may not comprise at least one CpG site before conversion) irrespective of whether the C of the at least one CpG site was unmethylated or methylated prior to the conversion, i.e. of whether the C has been converted or not. In one case, the region of the single-strand of DNA the oligonucleotide anneals to does not comprise any CpG sites (before and after conversion) and the oligonuclotide is methylation-unspecific solely for this reason. While a methylation-unspecific oligonucleotide may cover one or more CpG dinucleotides, it does so with mismatches and/or spacers. The term “mismatch” as used herein refers to base-pair mismatch in DNA, more specifically a base-pair that is unable to form normal base-pairing interactions (i.e., other than “A” with “T” or “U”, or “G” with “C”).

An oligonucleotide, i.e. a probe, blocker or primer, may also cover an SNP site with an SNP-unspecific mismatch or with a spacer.

The term “SNP site” as used herein refers to the site of an “SNP”, i.e. a single nucleotide polymorphism at a particular position in the, preferably human, genome that varies among a population of individuals. SNPs of the genomic DNA the present application refers to are known in the art and can be found in online databases such as dbSNP of NCBI (http://www.ncbi.nlm.nih.gov/snp).

The term “SNP-unspecific mismatch” as used herein refers to a mismatch that is due to a nucleotide substitution that does not substitute the nucleotide with one that corresponds to a nucleotide that is found at the same position in the genome of another individual of the same population.

The term “spacer” as used herein refers to a non-nucleotide spacer molecule, which increases, when joining two nucleotides, the distance between the two nucleotides to about the distance of one nucleotide (i.e. the distance the two nucleotides would be apart if they were joined by a third nucleotide). Non-limiting examples for spacers are Inosine, d-Uracil, halogenated bases, Amino-dT, C3, C12, Spacer 9, Spacer 18, and dSpacer)

The term “reflects” as used herein is to be understood to mean “is a result of” or “shows”.

The phrase “method for detecting the presence or absence of cancer in a subject” as used herein refers to a determination whether the subject has cancer or not. As will be understood by persons skilled in the art, such assessment normally may not be correct for 100% of the subjects, although it preferably is correct. The term, however, requires that a correct indication can be made for a statistically significant part of the subjects. Whether a part is statistically significant can be determined easily by the person skilled in the art using several well known statistical evaluation tools, for example, determination of confidence intervals, determination of p values, Student's t-test, Mann-Whitney test, etc. Details are provided in Dowdy and Wearden, Statistics for Research, John Wiley & Sons, New York 1983. The preferred confidence intervals are at least 50%, at least 60%, at least 70%, at least 80%, at least 90%, at least 95%. The p values are preferably 0.05, 0.01, or 0.005.

The term “risk thereof” with respect to the method for detecting the presence or absence of cancer in a subject refers to the detection of an increased risk of developing the cancer or an increased probability of having it. If the subject already has an increased risk in view of one or more risk factors that can be attributed to it (as defined herein), the ‘risk thereof’ refers to a risk that is increased further, i.e. that is in addition to the risk due to those risk factors.

The term “colon cancer” or “colorectal cancer” is used in the broadest sense and refers to (1) all stages and all forms of cancer arising from epithelial cells of the large intestine and/or rectum and/or (2) all stages and all forms of cancer affecting the lining of the large intestine and/or rectum. It includes the subtypes adenocarcinoma, gastrointestinal carcinoid tumor, gastrointestinal stromal tumor, primary colorectal lymphoma, leiomyosarcoma, melanoma or squamous cell carcinoma, each originating from the colon (colon cancer) or the rectum (rectal cancer). In the staging systems used for classification of colorectal cancer, the colon and rectum are treated as one organ. It also includes the following stages (as defined by the corresponding TNM classification(s) in brackets): stage 0 (Tis, N0, M0), stage I (T1-T2, N0, M0), stage IIA (T3, N0, M0), stage IIB (T4a, N0, M0), stage IIC (T4b, N0, M0), stage IIIA (T1-T2, N1, M0; or T1, N2a, M0), stage IIIB (T3-T4a, N1, M0; T2-T3, N2a, M0; or T1-T2, N2b, M0), stage IIIC (T4a, N2a, M0; T3-T4a, N2b, M0; or T4b N1-N2 M0), and stage IVA (any T, any N, M1a), stage IVA (any T, any N, M1b).

The TNM classification is a staging system for malignant cancer. As used herein the term “TNM classification” refers to the 6^{th} edition of the TNM stage grouping as defined in Sobin et al. (International Union Against Cancer (UICC), TNM Classification of Malignant tumors, 6^{th} ed. New York; Springer, 2002, pp. 191-203).

The term “cancer cell” as used herein refers to a cell that acquires a characteristic set of functional capabilities during their development, particularly one or more of the following: the ability to evade apoptosis, self-sufficiency in growth signals, insensitivity to anti-growth signals, tissue invasion/metastasis, significant growth potential, and/or sustained angiogenesis. The term is meant to encompass both pre-malignant and malignant cancer cells.

The term “tumor DNA” or “tumor DNA of a cancer cell” as used herein refers simply to DNA of a cancer cell. It is used only to distinguish DNA of a cancer cell more clearly from other DNA referred to herein. Thus, unless ambiguities are introduced, the term “DNA of a cancer cell” may be used instead.

The term “subject” as used herein refers to an individual, such as a mammal, including a non-human primate (e.g. chimpanzees and other apes and monkey species). Preferably it is a human. The term does not denote a particular age or sex. In principle, the subject can be any subject of which the methylation status within genomic DNA having a sequence comprised in SEQ ID NO: 11 and/or within genomic DNA having a sequence comprised in SEQ ID NO: 31, in particular from a sample comprising cell-free DNA from blood or a sample derived therefrom of a subject, is not known. Depending on what the method of the first aspect is to be used for, the term “subject” may have different limitations. For example, it the method is to be used for detecting cancer or screening subjects for cancer, the subject is not known to have cancer, i.e. it may or may not have cancer. In this example, the subject preferably is at risk or increased risk or is suspected to have cancer. “At risk or increased risk” means that one or more risk factors can be attributed to the subject), preferably as defined by the American Cancer Society generally or for the respective cancer.

The term “is indicative for” or “indicates” as used herein refers to an act of identifying or specifying the thing to be indicated. As will be understood by persons skilled in the art, such assessment normally may not be correct for 100% of the subjects, although it preferably is correct. The term, however, requires that a correct indication can be made for a statistically significant part of the subjects. For a description of statistic significance and suitable confidence intervals and p values, see above.

The term “amplifying” or “generating an amplicon” as used herein refers to amplifying a defined region of a double-stranded or single-stranded DNA template, typically with a polymerase chain reaction (PCR). An “amplicon” is a double-stranded fragment of DNA according to said defined region.

The term “pair of primers” as used herein refers to two oligonucleotides, namely a forward and a reverse primer, that have, with respect to a double-stranded nucleic acid molecule, sequences that are (at least substantially) identical to one strand each such that they each anneal to the complementary strand of the strand they are (at least substantially) identical to. The term “forward primer” refers to the primer which is (at least substantially) identical to the forward strand (as defined by the direction of the genomic reference sequence) of the double-stranded nucleic acid molecule, and the term “reverse primer” refers to the primer which is (at least substantially) identical to the reverse complementary strand of the forward strand in the double-stranded nucleic acid molecule. The distance between the sites where forward and reverse primer anneal to their template depends on the length of the amplicon the primers are supposed to allow generating. Typically, with respect to the present invention it is between 40 and 1000 bp, preferably between 40 and 200 bp and more preferably between 60 and 150 bp. Preferred amplicon sizes are specified herein. In case of single-stranded DNA template that is to be amplified using a pair of primers, only one of the primers anneals to the single strand in the first amplification cycle. The other primer then binds to the newly generated complementary strand such that the result of amplification is a double-stranded DNA fragment. The phrase “pair of primers suitable for generating an amplicon from a single strand of genomic DNA in which cytosine unmethylated in the 5-position has been converted to uracil or another base that does not hybridize to guanine” refers to a pair of primers which takes into account a base change from unmethylated cytosines to uracil, which basepairs with adenine and is therefore replaced with thymine in the amplicon.

The term “diagnosis” as used herein refers to a determination whether a subject does or does not have cancer, and preferably also which cancer. A diagnosis by methylation analysis of the target DNA as described herein may be supplemented with a further means as described herein to and/or narrow down the cancer detected with the methylation analysis. As will be understood by persons skilled in the art, the diagnosis normally may not be correct for 100% of the subjects, although it preferably is correct. The term, however, requires that a correct diagnosis can be made for a statistically significant part of the subjects. For a description of statistic significance and suitable confidence intervals and p values, see above.

The term “monitoring” as used herein refers to the repeated detection of methylated DNA or during a certain period of time, typically during at least 1 month, 6 months, 1 year, 2 years, 3 years, 5 years, 10 years, or any other period of time. For a subject having cancer, it is preferably detected at least throughout the time the subject is treated. Methylation may be detected based on the amount of methylated target DNA, particular based on changes in the amount in any type of periodical time segment, determined e.g., daily or at least once per week, month, or year. As will be understood by persons skilled in the art, the result of the monitoring normally may not be correct for 100% of the subjects, although it preferably is correct. The term, however, requires that a correct result of the monitoring can be achieved for a statistically significant part of the subjects. For a description of statistic significance and suitable confidence intervals and p values, see above.

The phrase “screening of subjects” refers to the use of the method of the first aspect with samples of a population of subjects. Preferably, the subjects have an increased risk for or are suspected of having CRC. In particular, one or more of the following risk factors recited herein can be attributed to the subjects of the population. In a specific embodiment, the same one or more risk factors can be attributed to all subjects of the population. For example, the population may be characterized by a certain minimal age (e.g. 50 or older). It is to be understood that the term “screening” does not necessarily indicate a definite diagnosis, but is intended to indicate an increased possibility of the presence or of the absence of CRC. An indicated increased possibility is preferably confirmed and/or narrowed down using a further means as described herein. As will be understood by persons skilled in the art, the screening result normally may not be correct for 100% of the subjects, although it preferably is correct. The term, however, requires that a correct screening result can be achieved for a statistically significant part of the subjects. For a description of statistic significance and suitable confidence intervals and p values, see above.

The term “treatment” or “treating” with respect to cancer as used herein refers to a therapeutic treatment, wherein the goal is to reduce progression of cancer. Beneficial or desired clinical results include, but are not limited to, release of symptoms, reduction of the length of the disease, stabilized pathological state (specifically not deteriorated), slowing down of the disease's progression, improving the pathological state and/or remission (both partial and total), preferably detectable. A successful treatment does not necessarily mean cure, but it can also mean a prolonged survival, compared to the expected survival if the treatment is not applied. In a preferred embodiment, the treatment is a first line treatment, i.e. the cancer was not treated previously. Cancer treatment involves a treatment regimen.

The term “treatment regimen” as used herein refers to how the subject is treated in view of the disease and available procedures and medication. Non-limiting examples of cancer treatment regimes are chemotherapy, surgery and/or irradiation or combinations thereof. The early detection of cancer the present invention enables allows in particular for a surgical treatment, especially for a curative resection. In particular, the term “treatment regimen” refers to administering one or more anti-cancer agents or therapies as defined below. The term “anti-cancer agent or therapy” as used herein refers to chemical, physical or biological agents or therapies, or surgery, including combinations thereof, with antiproliferative, antioncogenic and/or carcinostatic properties.

A chemical anti-cancer agent or therapy may be selected from the group consisting of alkylating agents, antimetabolites, plant alkaloyds and terpenoids and topoisomerase inhibitors. Preferably, the alykylating agents are platinum-based compounds. In one embodiment, the platinum-based compounds are selected from the group consisting of cisplatin, oxaliplatin, eptaplatin, lobaplatin, nedaplatin, carboplatin, iproplatin, tetraplatin, lobaplatin, DCP, PLD-147, JMI 18, JM216, JM335, and satraplatin.

A physical anti-cancer agent or therapy may be selected from the group consisting of radiation therapy (e.g. curative radiotherapy, adjuvant radiotherapy, palliative radiotherapy, teleradiotherapy, brachytherapy or metabolic radiotherapy), phototherapy (using, e.g. hematoporphoryn or photofrin II), and hyperthermia.

Surgery may be a curative resection, palliative surgery, preventive surgery or cytoreductive surgery. Typically, it involves an excision, e.g. intracapsular excision, marginal, extensive excision or radical excision as described in Baron and Valin (Rec. Med. Vet, Special Canc. 1990; 11 (166): 999-1007).

A biological anti-cancer agent or therapy may be selected from the group consisting of antibodies (e.g. antibodies stimulating an immune response destroying cancer cells such as retuximab or alemtuzubab, antibodies stimulating an immune response by binding to receptors of immune cells an inhibiting signals that prevent the immune cell to attack “own” cells, such as ipilimumab, antibodies interfering with the action of proteins necessary for tumor growth such as bevacizumab, cetuximab or panitumumab, or antibodies conjugated to a drug, preferably a cell-killing substance like a toxin, chemotherapeutic or radioactive molecule, such as Y-ibritumomab tiuxetan, I-tositumomab or ado-trastuzumab emtansine), cytokines (e.g. interferons or interleukins such as INF-alpha and IL-2), vaccines (e.g. vaccines comprising cancer-associated antigens, such as sipuleucel-T), oncolytic viruses (e.g. naturally oncolytic viruses such as reovirus, Newcastle disease virus or mumps virus, or viruses genetically engineered viruses such as measles virus, adenovirus, vaccinia virus or herpes virus preferentially targeting cells carrying cancer-associated antigens such as EGFR or HER-2), gene therapy agents (e.g. DNA or RNA replacing an altered tumor suppressor, blocking the expression of an oncogene, improving a subject's immune system, making cancer cells more sensitive to chemotherapy, radiotherapy or other treatments, inducing cellular suicide or conferring an anti-angiogenic effect) and adoptive T cells (e.g. subject-harvested tumor-invading T-cells selected for antitumor activity, or subject-harvested T-cells genetically modified to recognize a cancer-associated antigen).

In one embodiment, the one or more anti-cancer drugs is/are selected from the group consisting of Abiraterone Acetate, ABVD, ABVE, ABVE-PC, AC, AC-T, ADE, Ado-Trastuzumab Emtansine, Afatinib Dimaleate, Aldesleukin, Alemtuzumab, Aminolevulinic Acid, Anastrozole, Aprepitant, Arsenic Trioxide, Asparaginase Erwinia chrysanthemi, Axitinib, Azacitidine, BEACOPP, Belinostat, Bendamustine Hydrochloride, BEP, Bevacizumab, Bexarotene, Bicalutamide, Bleomycin, Bortezomib, Bosutinib, Brentuximab Vedotin, Busulfan, Cabazitaxel, Cabozantinib-S-Malate, CAFCapecitabine, CAPOX, Carboplatin, CARBOPLATIN-TAXOL, Carfilzomib, Carmustine, Carmustine Implant, Ceritinib, Cetuximab, Chlorambucil, CHLORAMBUCIL-PREDNISONE, CHOP, Cisplatin, Clofarabine, CMF, COPP, COPP-ABV, Crizotinib, CVP, Cyclophosphamide, Cytarabine, Cytarabine, Liposomal, Dabrafenib, Dacarbazine, Dactinomycin, Dasatinib, Daunorubicin Hydrochloride, Decitabine, Degarelix, Denileukin Diftitox, Denosumab, Dexrazoxane Hydrochloride, Docetaxel, Doxorubicin Hydrochloride, Doxorubicin Hydrochloride Liposome, Eltrombopag Olamine, Enzalutamide, Epirubicin Hydrochloride, EPOCH, Eribulin Mesylate, Erlotinib Hydrochloride, Etoposide Phosphate, Everolimus, Exemestane, FEC, Filgrastim, Fludarabine Phosphate, Fluorouracil, FU-LV, Fulvestrant, Gefitinib, Gemcitabine Hydrochloride, GEMCITABINE-CISPLATIN, GEMCITABINE-OXALIPLATIN, Gemtuzumab Ozogamicin, Glucarpidase, Goserelin Acetate, HPV Bivalent Vaccine, Recombinant HPV Quadrivalent Vaccine, Hyper-CVAD, Ibritumomab Tiuxetan, Ibrutinib, ICE, Idelalisib, Ifosfamide, Imatinib, Mesylate, Imiquimod, Iodine 131 Tositumomab and Tositumomab, Ipilimumab, Irinotecan Hydrochloride, Ixabepilone, Lapatinib Ditosylate, Lenalidomide, Letrozole, Leucovorin Calcium, Leuprolide Acetate, Liposomal Cytarabine, Lomustine, Mechlorethamine Hydrochloride, Megestrol Acetate, Mercaptopurine, Mesna, Methotrexate, Mitomycin C, Mitoxantrone Hydrochloride, MOPP, Nelarabine, Nilotinib, Obinutuzumab, Ofatumumab, Omacetaxine Mepesuccinate, OEPA, OFF, OPPA, Oxaliplatin, Paclitaxel, Paclitaxel Albumin-stabilized Nanoparticle Formulation, PAD, Palifermin, Palonosetron Hydrochloride, Pamidronate Disodium, Panitumumab, Pazopanib Hydrochloride, Pegaspargase, Peginterferon Alfa-2b, Pembrolizumab, Pemetrexed Disodium, Pertuzumab, Plerixafor, Pomalidomide, Ponatinib Hydrochloride, Pralatrexate, Prednisone, Procarbazine Hydrochloride, Radium 223 Dichloride, Raloxifene Hydrochloride, Ramucirumab, Rasburicase, R-CHOP, R-CVP, Recombinant HPV Bivalent Vaccine, Recombinant HPV Quadrivalent Vaccine, Recombinant Interferon Alfa-2b, Regorafenib, Rituximab, Romidepsin, Romiplostim, Ruxolitinib Phosphate, Siltuximab, Sipuleucel-T, Sorafenib Tosylate, STANFORD V, Sunitinib Malate, TAC, Talc, Tamoxifen Citrate, Temozolomide, Temsirolimus, Thalidomide, Topotecan Hydrochloride, Toremifene, Tositumomab and I 131 Iodine Tositumomab, TPF, Trametinib, Trastuzumab, Vandetanib, VAMP, VelP, Vemurafenib, Vinblastine Sulfate, Vincristine Sulfate, Vincristine Sulfate Liposome, Vinorelbine Tartrate, Vismodegib, Vorinostat, XELOX, Ziv-Aflibercept, and Zoledronic Acid.

### SEQ IDs Referred to in the Application

The present application refers to SEQ ID NOs 1-57. An overview and explanation of these SED IDs is given in the following Table 1.

TABLE 1
SEQ IDs of the specification. rc means reverse complement, C
to T or G to A means converted by bisulfite conversion of cytosines
outside of CpG context into uracil and replaced by thymidine
in subsequent amplification. bis1 refers to the bisulfite converted
forward strand (as recited in the SEQ ID of the respective genomic
DNA) and bis2 to the bisulfite converted reverse complement
strand of the forward strand (reverse complement of the SEQ
ID of the respective genomic DNA), whereby the direction of
the strand is defined by the direction of the genomic reference
sequence as e.g. obtained from the genome build (HCGR38). For
a mapping of the sequences, see FIG. 1.
ANKRD13BoB1 Assay (associated with
ANKRD13B) 17: 29613595-29613658

| | |
|---|---|
| SEQ ID NO: 1 | genomic reference |
| SEQ ID NO: 2 | C to T (bis1) |
| SEQ ID NO: 3 | rc C to T (bis1) |
| SEQ ID NO: 4 | G to A (bis2 rc) |
| SEQ ID NO: 5 | G to A (bis2 rc) rc |

ANKRD13B ExtendedAssayRegionA
17: 29613595-29613700

| | |
|---|---|
| SEQ ID NO: 6 | genomic reference |
| SEQ ID NO: 7 | C to T (bis1) |
| SEQ ID NO: 8 | rc C to T (bis1) |
| SEQ ID NO: 9 | G to A (bis2 rc) |
| SEQ ID NO: 10 | G to A (bis2 rc) rc |

ANKRD13B ExtendedAssayRegionB
17: 29613595-29613940

| | |
|---|---|
| SEQ ID NO: 11 | genomic reference |
| SEQ ID NO: 12 | C to T (bis1) |
| SEQ ID NO: 13 | rc C to T (bis1) |
| SEQ ID NO: 14 | G to A (bis2 rc) |
| SEQ ID NO: 15 | G to A (bis2 rc) rc |

ANKRD13BoB2 Assay (associated with
ANKRD13B) 17: 29613595-29613681

| | |
|---|---|
| SEQ ID NO: 16 | genomic reference |
| SEQ ID NO: 17 | C to T (bis1) |
| SEQ ID NO: 18 | rc C to T (bis1) |
| SEQ ID NO: 19 | G to A (bis2 rc) |
| SEQ ID NO: 20 | G to A (bis2 rc) rc |

SEPTIN9oB1 Assay (associated with
SEPTIN9) 17: 77373475-77373533

| | |
|---|---|
| SEQ ID NO: 21 | genomic reference |
| SEQ ID NO: 22 | C to T (bis1) |
| SEQ ID NO: 23 | rc C to T (bis1) |
| SEQ ID NO: 24 | G to A (bis2 rc) |
| SEQ ID NO: 25 | G to A (bis2 rc) rc |

SEPTIN9 AssayRegion 17:
77373475-77373549

| | |
|---|---|
| SEQ ID NO: 26 | genomic reference |
| SEQ ID NO: 27 | C to T (bis1) |
| SEQ ID NO: 28 | rc C to T (bis1) |
| SEQ ID NO: 29 | G to A (bis2 rc) |
| SEQ ID NO: 30 | G to A (bis2 rc) rc |

SEPTIN9 Assay + CpG-island 17:
77372606-77374424

| | |
|---|---|
| SEQ ID NO: 31 | genomic reference |
| SEQ ID NO: 32 | C to T (bis1) |
| SEQ ID NO: 33 | rc C to T (bis1) |
| SEQ ID NO: 34 | G to A (bis2 rc) |
| SEQ ID NO: 35 | G to A (bis2 rc) rc |

SEPTIN9 ExtendedAssayRegion
17: 77372979-77374040

| | |
|---|---|
| SEQ ID NO: 36 | genomic reference |
| SEQ ID NO: 37 | C to T (bis1) |
| SEQ ID NO: 38 | rc C to T (bis1) |
| SEQ ID NO: 39 | G to A (bis2 rc) |
| SEQ ID NO: 40 | G to A (bis2 rc) rc |

SEPTIN9oB2 Assay (associated with
SEPTIN9) 17: 77373482-77373549

| | |
|---|---|
| SEQ ID NO: 41 | genomic reference |
| SEQ ID NO: 42 | C to T (bis1) |
| SEQ ID NO: 43 | rc C to T (bis1) |
| SEQ ID NO: 44 | G to A (bis2 rc) |
| SEQ ID NO: 45 | G to A (bis2 rc) rc |
| SEQ ID NO: 46 | CO6B1-F4 |
| SEQ ID NO: 47 | CO6B1-R6 |
| SEQ ID NO: 48 | CO6B2-F3 |
| SEQ ID NO: 49 | CO6B2-R5 |
| SEQ ID NO: 50 | S9B1-F3 |
| SEQ ID NO: 51 | S9B1-R1 |
| SEQ ID NO: 52 | S9B2-F3 |
| SEQ ID NO: 53 | S9B2-R11 |
| SEQ ID NO: 54 | CO6B1-P4 |
| SEQ ID NO: 55 | CO6B2-P1 |
| SEQ ID NO: 56 | S9B1-P2 |
| SEQ ID NO: 57 | S9B2-P8 |

The invention is described by way of the following examples which are to be construed as merely illustrative and not limitative of the scope of the invention.

### Example 1

Blood plasma and urine samples from colorectal carcinoma patients (CRC) and individuals with no evidence of disease (NED) were processed with the Epi BiSKit (Epigenomics AG). Briefly, DNA extraction from plasma samples and bisulfite conversion of DNA was performed with the Epi BiSKit Plasma Kit according to the workflow as defined in the instructions for use (IFU) of the Epi BiSKit (Epigenomics AG), urine samples were treated as blood plasma samples.

For each sample, 15 μl bisulfite treated DNA were amplified in triplicate in a Real-time PCR pentaplex containing four methylation sensitive (MSP) assays, and one bisulfite specific and methylation unspecific ACTB control assay (see FIG. 1). The assays were measured using an Applied Biosystems 7500 Fast Dx Real-Time PCR Instrument. As a quality measure, data from PCR pentaplex reactions in triplicate representing one sample with at least one reaction providing no ACTB control assay curve calls or ACTB Cts>=32 were removed from the data set due to too low DNA levels. Ct curve calls for marker assays <20 were removed from the data set too, as no amplification with such early cycle thresholds is expected.

In order to demonstrate that different ways to assess the data for showing discriminatory effects, the following methods were used:
1. The minimum Cts for all four MSP assays over triplicates were used in logistic regression. The area under the curve (AUC) of Receiver operating characteristic (ROC) analysis was used to assess the discrimination of CRC vs. NED.
2. Curves were classified as positive or negative based on Ct cutoffs (42 for plasma, 39 for urine). Performance was assessed by ROC analysis using 13 Sensitivity/Specificity data pairs for N₁ [0;12]/12 (12 due to the four methylation sensitive assays in triplicates) positive calls.
3. Curves were classified based on Ct cutoffs as above. Each of the four MSP assays in triplicate was classified as positive measurement if two of the three curve calls were positive. Performance was assessed by ROC analysis using 5 Sensitivity/Specificity data point pairs for N₂ [0;4]/4 positive assay calls.
4. (Plasma only): Curves were classified as positive or negative based on a Ct cutoff 42. Sample diagnosis was classified as positive if at least one of the following conditions was met: one of the four assays was positive in all 3 replicates, two of the four assays were positive in at least 2 replicates.

### Plasma Results

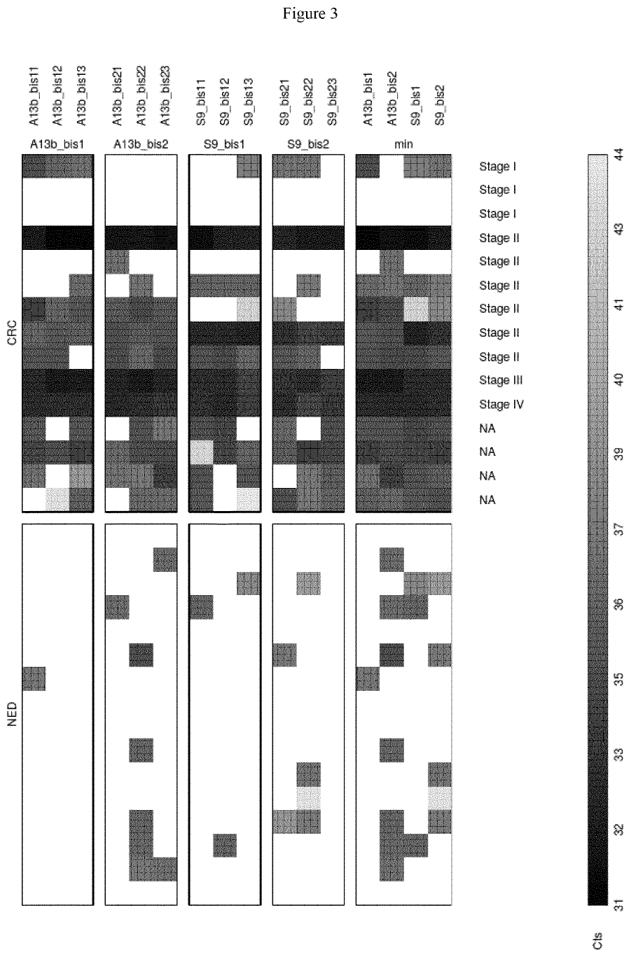

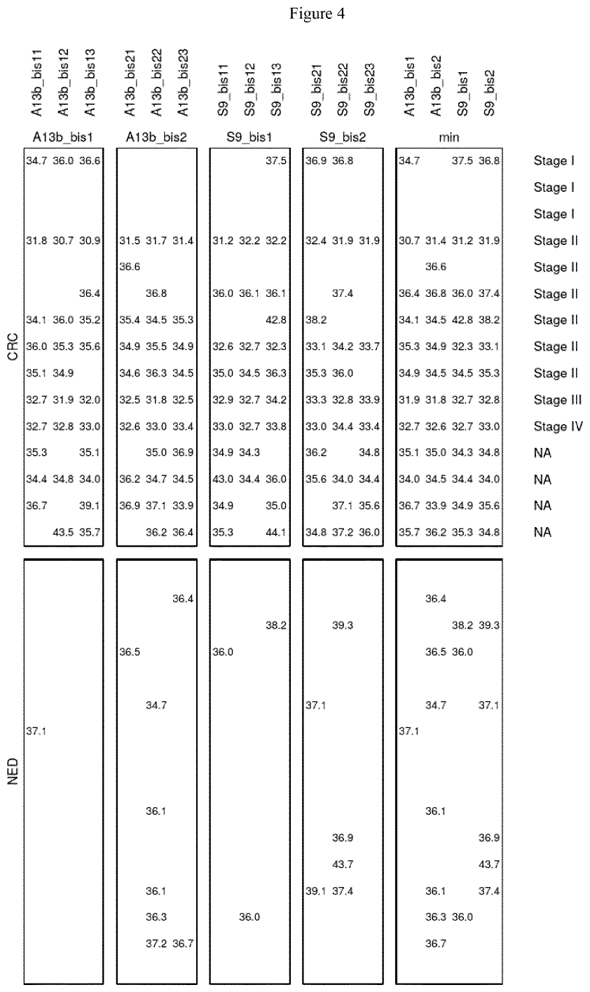

From 20 CRC and 20 NED samples, 8 dropped out due to low ACTB and one due to a marker cycle threshold Ct below 20—the final data set for plasma samples comprised 15 CRC and 16 NED (see FIG. 3 and FIG. 4).

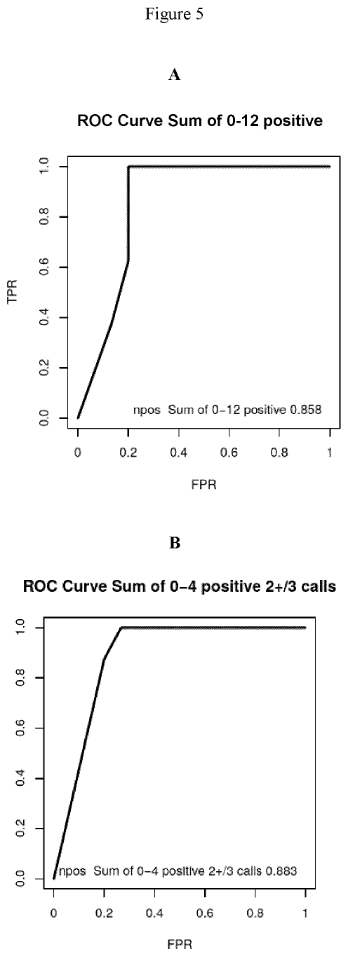

The Realtime-PCR measurements as assessed by minimum Cts (method 1) for all four MSP marker assays lead to an AUC of 0.85. The assessment by N₁/12 binary calls (method 2) lead to AUC of 0.86 (see FIG. 5A). The assessment by N₂/4 at least double positive assays in triplicate (method 3) lead to an AUC of 0.88 (see FIG. 5 B). The performance by the method 4 (see Table 2) lead to Sensitivity of 0.8 at Specificity of 1.0 (see Table 3).

TABLE 2
Confusion matrix for results from CRC and
NED plasma samples assessed with method 4.

| | negative | positive |
|---|---|---|
| NED | 16 | 0 |
| CRC | 3 | 12 |

TABLE 3
Sensitivity and Specificity and 95% confidence intervals for
plasma samples from CRC and NED assessed with method 4.

| | | |
|---|---|---|
| Sensitivity | 0.8 | CI 95 [0.55-0.93] |
| Specificity | 1.0 | CI 95 [0.81-1.00] |

### Urine Results

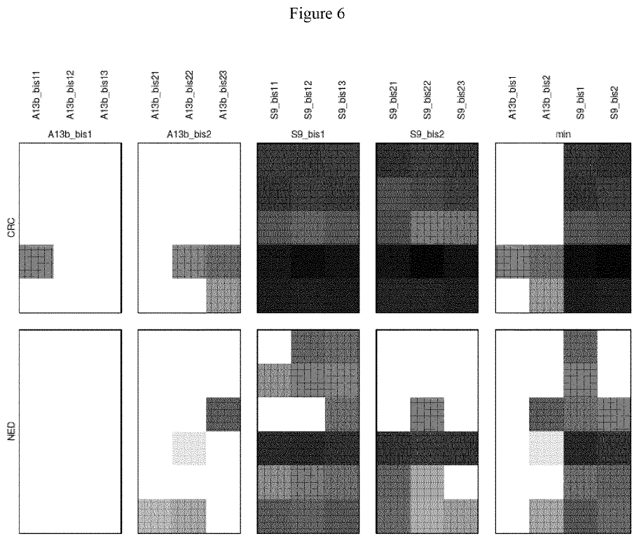

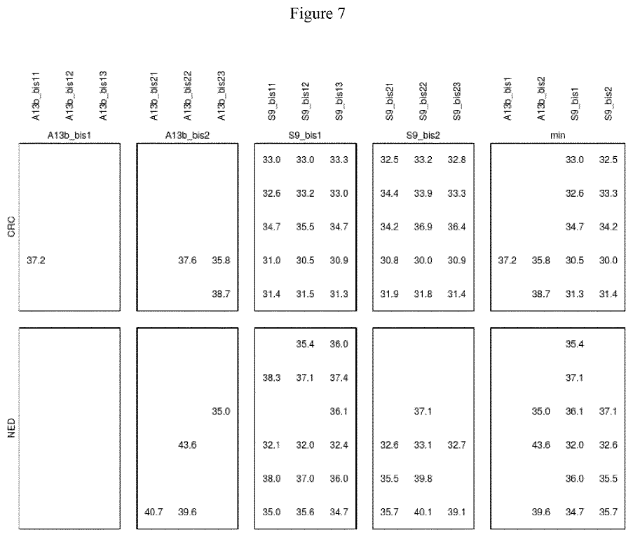

From 6 CRC and 6 NED urines one sample dropped out due to low ACTB, leaving a data set of 5 CRC vs. 6 NED. Quantitative assessment of min Cts in a logistic regression (method 1) yielded a ROC curve with AUC of 1.0. Discrimination by later Cts in NEDs was observed over the whole set (see FIG. 6 and FIG. 7).

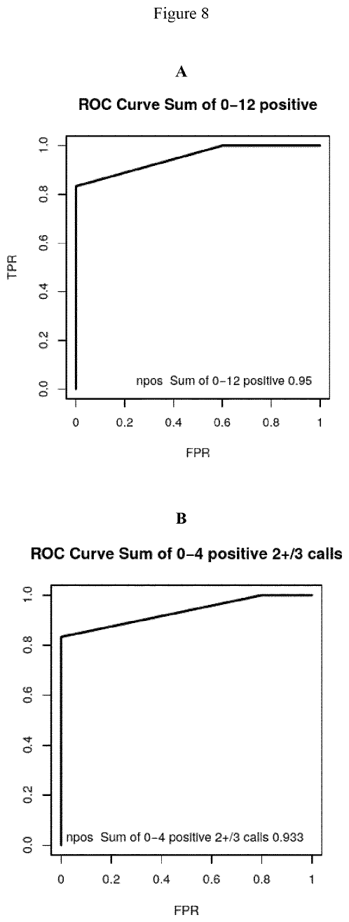

The Realtime-PCR measurements as assessed by minimum Cts for all 4 marker assays (method 1) lead to an AUC of 1.0. The assessment by N₁/12 binary calls (method 2) lead to AUC of 0.95 (see FIG. 8A). The assessment by N₂/4 at least double positive assays in triplicate (method 3) lead to an AUC of 0.93 (see FIG. 8 B).

1. A method for detecting DNA methylation (i) at one or more CpG dinucleotides within genomic DNA having a sequence comprised in SEQ ID NO: 11 (mANKRD13B target region) and optionally (ii) at one or more CpG dinucleotides within genomic DNA having a sequence comprised in SEQ ID NO: 31 (mSEPTIN9 target region) in a biological sample of a subject.

2. The method of claim 1, comprising the steps of
(a) converting cytosine unmethylated in the 5-position to uracil or another base that does not hybridize to guanine in the genomic DNA of the biological sample; and
(b) detecting DNA methylation within the mANKRD13B target region by detecting unconverted cytosine in converted DNA according to SEQ ID NOs 12 and 15, and optionally detecting DNA methylation within the mSEPTIN9 target region by detecting unconverted cytosine in converted DNA according to SEQ ID NOs 32 and 35,
preferably wherein methylation of at least one, more preferably each, of the one or more CpG dinucleotides within the mANKRD13B target region is detected on both the sense strand and the anti-sense strand, and optionally wherein methylation of at least one, more preferably each, of the one or more CpG dinucleotides within the mSEPTIN9 target region is detected on both the sense strand and the anti-sense strand.

3. The method of claim 1, wherein detecting DNA methylation comprises a PCR using at least one methylation-specific primer.

4. The method of claim 1, wherein detecting DNA methylation comprises a multiplex real-time PCR comprising
(1) primers suitable for amplifying DNA within SEQ ID NO: 12,
(2) primers suitable for amplifying DNA within SEQ ID NO: 15,
(3) primers suitable for amplifying DNA within SEQ ID NO: 32, and
(4) primers suitable for amplifying DNA within SEQ ID NO: 35, and
optionally (5) methylation-unspecific primers suitable for amplifying control DNA, wherein the primers of (1) to (4) preferably are methylation-specific primers.

5. The method of claim 1, wherein DNA methylation is detected at 7 or more CpG dinucleotides within the mANKRD13B target region.

6. The method of claim 1, wherein the biological sample is a colon or rectum tissue sample or a liquid biopsy, preferably a blood sample, a blood-derived sample, a urine sample, a urine-derived sample, a saliva sample, or a saliva-derived sample.

7. The method of claim 1, wherein the subject is suspected of having CRC, has an increased risk of developing CRC, has had CRC, or has CRC.

8. The method of claim 1, wherein the genomic DNA having a sequence comprised in SEQ ID NO: 11 has a sequence comprised in SEQ ID NO: 6, preferably in SEQ ID NO: 16, more preferably in SEQ ID NO: 1, and/or wherein the genomic DNA having a sequence comprised in SEQ ID NO: 31 has a sequence comprised in SEQ ID NO: 36, preferably in SEQ ID NO: 26, more preferably in SEQ ID NO: 21 or SEQ ID NO: 41.

9. A method for detecting colorectal cancer (CRC) in a subject, comprising detecting DNA methylation according to claim 1, wherein
(1), if the mANKRD13B target region and not the mSEPTIN9 target region is examined, the presence of detected methylated genomic DNA indicates the presence of CRC, and
(2), if both the mANKRD13B target region and the mSEPTIN9 target region are examined, the presence of detected methylated genomic DNA of at least one of the target regions indicates the presence of CRC.

10. A method for monitoring a subject suspected of having CRC, having an increased risk of developing colorectal cancer (CRC), or who has had CRC, comprising detecting DNA methylation according to claim 9 repeatedly, wherein
(1), if the mANKRD13B target region and not the mSEPTIN9 target region is examined, the presence of detected methylated genomic DNA indicates the presence of CRC and the absence of detected methylated genomic DNA indicates the absence of CRC, and
(2), if both the mANKRD13B target region and the mSEPTIN9 target region are examined, the presence of detected methylated genomic DNA of at least one of the target regions indicates the presence of CRC.

11. A kit comprising (i) a primer pair consisting of a forward primer oligonucleotide comprising a sequence that is substantially identical to a stretch of contiguous nucleotides of SEQ ID NO: 12 or 15, and a reverse primer oligonucleotide comprising a sequence that is substantially identical to a stretch of contiguous nucleotides of SEQ ID NO: 13 or 14, respectively, wherein the primer pair is suitable for amplification of a sequence comprising one or more CpG dinucleotides of SEQ ID NO: 12 or 15, respectively, and optionally (ii) a primer pair consisting of a forward primer oligonucleotide comprising a sequence that is substantially identical to a stretch of contiguous nucleotides of SEQ ID NO: 32 or 35, and a reverse primer oligonucleotide comprising a sequence that is substantially identical to a stretch of contiguous nucleotides of SEQ ID NO: 33 or 34, respectively, wherein the primer pair is suitable for amplification of a sequence comprising one or more CpG dinucleotides of SEQ ID NO: 32 or 35, respectively.

12. The kit of claim 11, wherein the kit comprises:
(1) a first primer pair consisting of a first forward primer oligonucleotide comprising a sequence that is substantially identical to a stretch of contiguous nucleotides of SEQ ID NO: 12, and a first reverse primer oligonucleotide comprising a sequence that is substantially identical to a stretch of contiguous nucleotides of SEQ ID NO: 13, wherein the primer pair is suitable for amplification of a sequence comprising one or more CpG dinucleotides of SEQ ID NO: 12, and
(2) a second primer pair consisting of a second forward primer oligonucleotide comprising a sequence that is substantially identical to a stretch of contiguous nucleotides of SEQ ID NO: 15, and a second reverse primer oligonucleotide comprising a sequence that is substantially identical to a stretch of contiguous nucleotides of SEQ ID NO: 14, wherein the primer pair is suitable for amplification of a sequence comprising one or more CpG dinucleotides of SEQ ID NO: 15.

13. The kit of claim 12, wherein the kit comprises
(3) a third primer pair consisting of a third forward primer oligonucleotide comprising a sequence that is substantially identical to a stretch of contiguous nucleotides of SEQ ID NO: 32, and a third reverse primer oligonucleotide comprising a sequence that is substantially identical to a stretch of contiguous nucleotides of SEQ ID NO: 33, wherein the primer pair is suitable for amplification of a sequence comprising one or more CpG dinucleotides of SEQ ID NO: 32, and
(4) a fourth primer pair consisting of a fourth forward primer oligonucleotide comprising a sequence that is substantially identical to a stretch of contiguous nucleotides of SEQ ID NO: 35, and a fourth reverse primer oligonucleotide comprising a sequence that is substantially identical to a stretch of contiguous nucleotides of SEQ ID NO: 34, wherein the primer pair is suitable for amplification of a sequence comprising one or more CpG dinucleotides of SEQ ID NO: 35, and
optionally (5) a fifth primer pair consisting of a methylation-unspecific fifth forward primer oligonucleotide and a methylation-unspecific fifth reverse primer oligonucleotide suitable for amplifying control DNA.

14. The kit of a claim 11, wherein the primer pair suitable for amplification of a sequence comprising one or more CpG dinucleotides of SEQ ID NO: 12 is suitable for amplification of a sequence comprising 7 or more CpG dinucleotides of SEQ ID NO: 12, and/or the primer pair suitable for amplification of a sequence comprising one or more CpG dinucleotides of SEQ ID NO: 15 is suitable for amplification of a sequence comprising 7 or more CpG dinucleotides of SEQ ID NO: 15.

15. Use of the method of claim 1, or of the kit for the detection of colorectal cancer (CRC), or for monitoring a subject having an increased risk of developing CRC, suspected of having CRC or that has had CRC.
